(19) United States
(12) Patent Application Publication
(10) Pub. No.: US 2021/0393810 A1
(43) Pub. Date: Dec. 23, 2021

(54) DIHYDRAZONE COMPOUNDS HAVING HIGH AFFINITY TO A BETA PROTEIN AND TAU PROTEIN, DERIVATIVES THEREOF AND USE THEREOF

(71) Applicants: HTA CO., LTD., Beijing (CN); BEIJING NORMAL UNIVERSITY, Beijing (CN)

(72) Inventors: Mengchao CUI, Beijing (CN); Langtao SHEN, Beijing (CN); Xuesong DENG, Beijing (CN); Kaixiang ZHOU, Beijing (CN)

(73) Assignees: HTA CO., LTD., Beijing (CN); BEIJING NORMAL UNIVERSITY, Beijing (CN)

(21) Appl. No.: 17/287,642

(22) PCT Filed: Jan. 2, 2020
(86) PCT No.: PCT/CN2020/070009

§ 371 Date: Apr. 22, 2021

(30) Foreign Application Priority Data
Jan. 4, 2019 (CN) 201910007703.X

(51) Int. Cl. A61K 51/04; C07C 251/88; C07D 295/135; C07C 255/66; C07D 213/74; C07D 213/64; C07D 317/58; A61K 49/00; A61P 25/28

(52) U.S. Cl. CPC A61K 51/0455; C07C 251/88; C07D 295/135; C07C 255/66; A61P 25/28; C07D 213/64; C07D 317/58; A61K 51/04; A61K 49/0052; C07D 213/74

(57) ABSTRACT

The present disclosure provides dihydrazone compounds having high affinity to Aβ protein and Tau protein and derivatives thereof. The structure of the compounds is shown by formula (I)

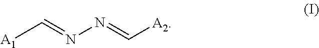

### CROSS REFERENCE

The disclosure claims the priority of Chinese Patent Application No. 201910007703.X filed on Jan. 4, 2019, entitled “DIHYDRAZONE COMPOUNDS HAVING HIGH AFFINITY TO Aβ PROTEIN AND TAU PROTEIN, DERIVATIVES THEREOF AND USE THEREOF”, the entire contents of which are incorporated in the disclosure by reference herein in their entirety.

### TECHNICAL FIELD

The present disclosure relates to the technical field of medical image, and in particular to, dihydrazone compounds having high affinity to Aβ protein and Tau protein, derivatives thereof and use thereof.

### BACKGROUND

Alzheimer's disease (AD) is the most common disease in senile dementia and its clinical manifestations are mainly progressive memory loss and irreversible impairment of perceptive function. It has become the fourth major disease that seriously threatens the health of the elderly after tumors, heart disease, stroke, and diabetes. The neuropathological studies of AD have shown that the deposition of senile plaques (SPs) occur mainly outside the cells in the cortex and hippocampus, intracellular neurofibrillary tangles (NFTs) and axon dystrophy (DNs) are important pathological feature of AD. Studies have shown that Aβ protein deposition in the brain has begun 5-10 years before the onset of AD. After aggregation and deposition, a series of neuronal apoptosis processes are initiated, such as inflammation, oxidative damage, and excessive neurotransmitter. The ultra-fine structure of neurofibrillary tangles is formed by the tight deposition of paired helical windings (PHF). In AD, tau protein aggregates into PHF, and will be further modified such as ubiquitination, glycosylation, nitration, polyamination and highly phosphorylated. Neurofibrillary tangles selectively kill neurons in specific areas of the brain, and are closely related to the severity of AD. Not only that, tangled Alzheimer's disease (TD), argyrophilic granular disease (AGD), progressive supranuclear palsy (PSP), cortical basal ganglia degeneration (CBD), Parkinson's syndrome (PD) are all closely related to neurofibrillary tangles in the brain. Therefore, it is of great significance for the quantification of Aβ and Tau protein levels in the brain of AD. At present, the most common clinical method is the analysis of cerebrospinal fluid, but this method is invasive and has certain surgical risks. Modern molecular imaging technologies such as positron emission tomography (PET) and single photon emission computed tomography (SPECT) scanning have the advantages of no damage, accurate diagnosis and monitoring of disease development, and have been widely recognized by clinical trials in recent years. Therefore, the development of molecular probes having high affinity and selectivity to Aβ and Tau proteins, with the use of medical imaging technology, can achieve early non-invasive diagnosis of AD, which is also of great significance for the monitoring and treatment of AD diseases.

In recent years, Aβ plaque imaging agents used for nuclear medicine PET or SPECT imaging have become relatively mature, and many probes have entered the clinical stage. For example, the representative compound of 2-phenylbenzothiazole [¹¹C]PIB (Mathis, C A et al. Journal of Medicinal Chemistry. 2003, 46: 2740-2754) is currently the most widely used Aβ plaque imaging agent, and its analogue [¹⁸F]GE-067 (Mathis, C. A. et al. Journal of Nuclear Medicine. 2007, 48:56p) has good clinical application prospects and has been approved by the US FDA; stilbene bistriazine derivative[¹⁸F]AV-45 (Chio, S. R et al. Journal of Nuclear Medicine. 2009, 50:1887-1894) is the first Aβ plaque imaging agent approved by the US FDA, [¹⁸F]BAY94-9172 (Zhang, W. et al. Nuclear Medicine and Biology. 2005, 32:799-809) is also now actively carrying out Phase III clinical trials. 2-Phenylimidazopyridine derivative [¹²³I]IMPY (Zhang, Z. P. et al. Journal of Medicinal Chemistry. 2003, 46:237-243) is the first SPECT imaging agent to enter the clinical stage, but cannot be used in practical applications due to its poor stability in vivo. Relative to the maturity of the study of Aβ plaque imaging agents, probes specifically targeting tau protein are in the development stage. [¹⁸F]THK-5117 (Harada, R et al. European Journal of Nuclear Medicine and Molecular Imaging. 2015, 42:1052-1061) in the THK series of compounds reported by Tohoku University in Japan and further improved [¹⁸F]THK-5351 (Harada, R et al. Journal of Nuclear Medicine. 2016, 57:208-214) have undergone clinical trials, and Siemens has also developed a probe [¹⁸F]T807 (Xia, C.-F et al. Alzheimer's Dementia. 2013, 9: 666-676) and [¹⁸F]T808 (Chien, D. T et al. Journal of Alzheimer's Dementia. 2014, 38: 171-184). [¹¹C]PBB3 (Maruyama, M et al. Neuron. 2013, 79:1-15) in the PBB series developed by the Institute of Radiology (NIRS) in Chiba, Japan, is not stable in vivo in human trials due to its cis-trans isomerism in the chemical structure. [¹⁸F]MK-6420 (Walji, A. M et al. Journal of Medicinal Chemistry. 2016, 59: 4778-4789) reported by Merck and its collaborators has also entered clinical evaluation. Roche also reported [¹¹C]RO6931643, [¹¹C]RO6924963, [¹⁸F]RO6958948 (Luca C. Gobbi et al. Journal of Medicinal Chemistry. 2017, 60: 7350-7370). Among these probes, [¹⁸F]T807 has been conducted the most in-depth study of AD cases. Although [¹⁸F]T807 has high selectivity to tau protein, the latest research has found that it has a slow clearance in the brain and a wide distribution of non-specific combination, resulting in a low signal-to-noise ratio. Therefore, it is urgent to develop Aβ and Tau probes with higher detection sensitivity and specificity to provide a strong technical support for the treatment of later AD diseases.

### SUMMARY

The purpose of the present disclosure is to provide dihydrazone compounds having high affinity to Aβ protein and Tau protein, and derivatives and use thereof.

In order to achieve the purpose of the present disclosure, in a first aspect, the present disclosure provides a dihydrazone compound having high affinity to Aβ protein and Tau protein, and a structure of the compound is shown in formula (I):

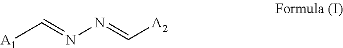

wherein, A₁ and A₂ each independently represent an aryl group or a substituted aryl group;

the aryl group or substituted aryl group is selected from

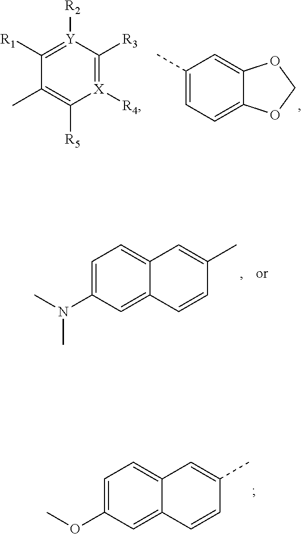

X and Y each independently represent C or N, wherein when X is N, R₄ does not exist, and when Y is N, R₂ does not exist; when X and Y are both N, R₂ and R4 do not exist;

R₁, R₂, R₃, R₄, and R₅ each independently represent H, ¹⁸F, F, Cl, Br, ¹²³I, ¹²⁴I, ¹²⁵I, I, CH₃, tBu, NO₂, CN, CF₃, OH, O¹¹CH₃, OCH₃, NH₂, NH¹¹CH₃, NHCH₃, N(CH₃)₂, N(CH₂CH₃)₂, N(Ph)₂,

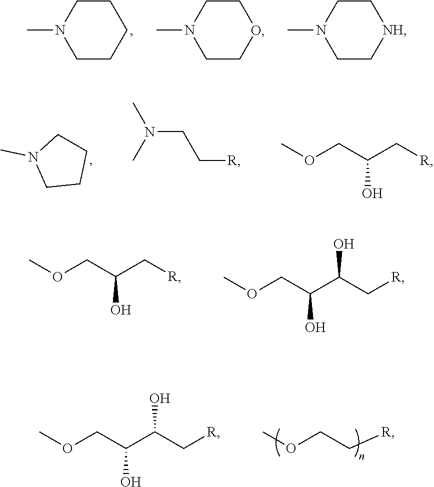

O(CH₂)ₘ¹⁸F, O(CH₂)ₘ¹⁹F; wherein R is OH, ¹⁸F or ¹⁹F, m and n are integers between 1 to 6.

Preferably, the compound is selected from any one of the following compounds 9, 11, 13-17, 36, 94, 63, and 87-93:

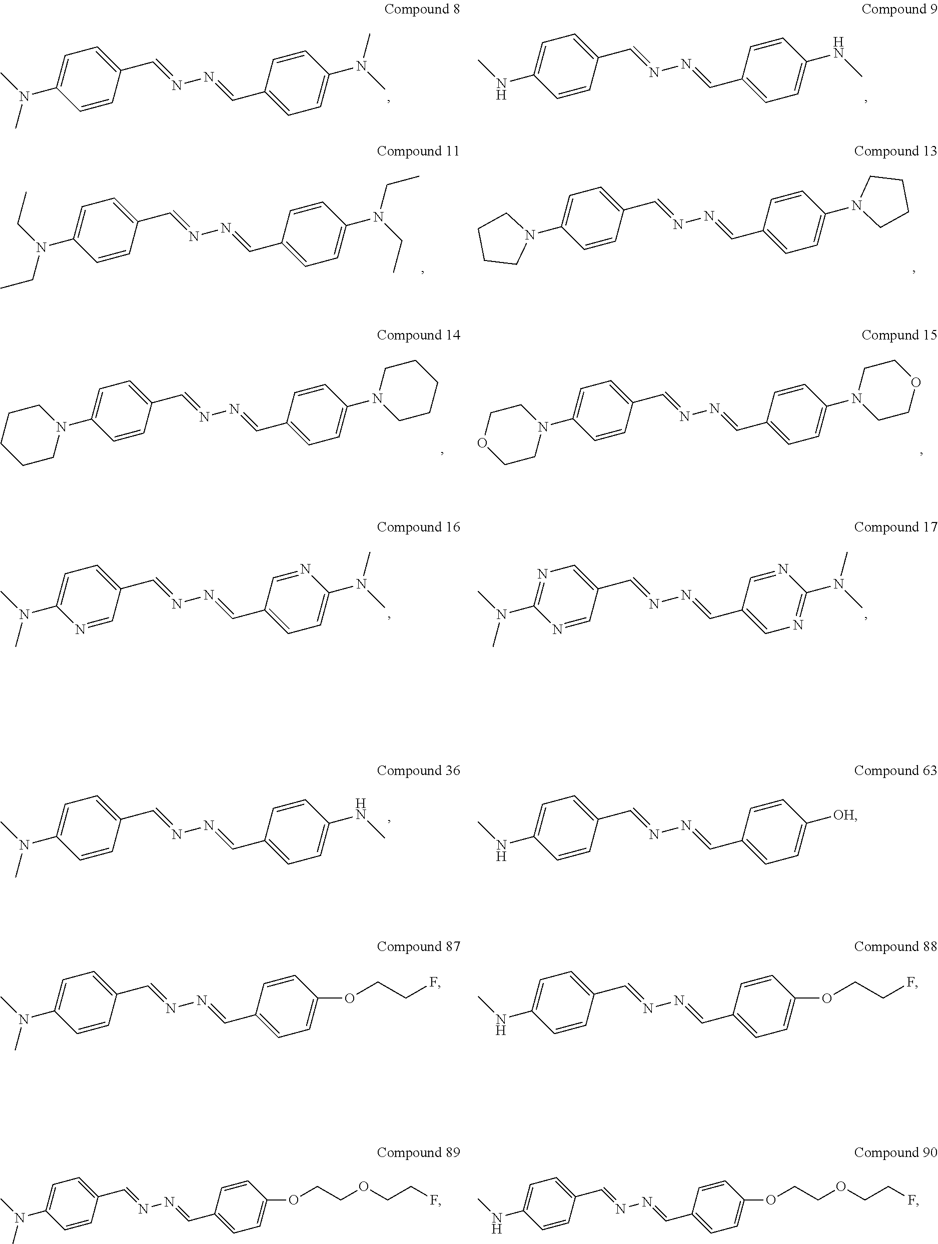

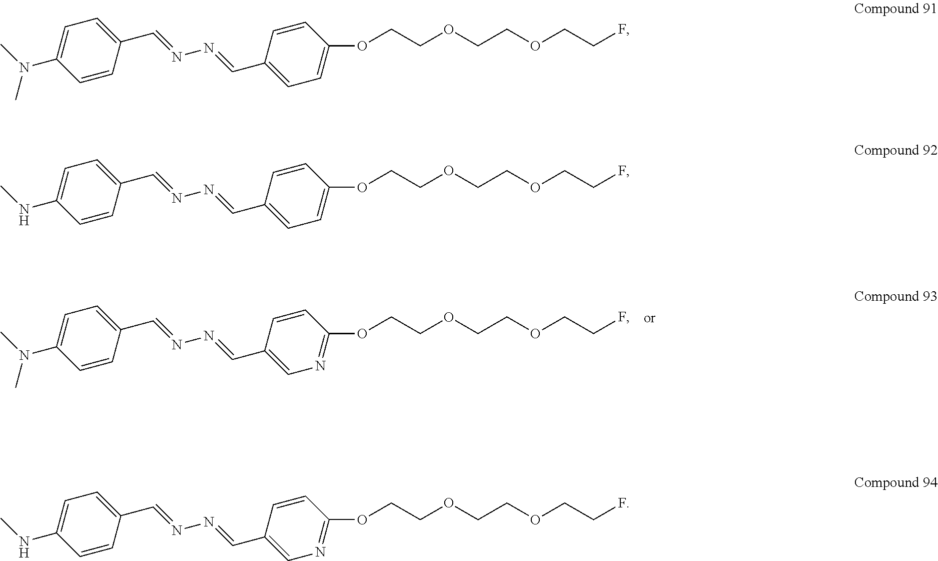 [SMILES: CN(C)c1ccc(/C=N/N=C/c2ccc(OCCOCCOCCF)cc2)cc1 ; CNc1ccc(/C=N/N=C/c2ccc(OCCOCCOCCF)cc2)cc1 ; CN(C)c1ccc(/C=N/N=C/c2ccc(OCCOCCOCCF)nc2)cc1 ; CNc1ccc(/C=N/N=C/c2ccc(OCCOCCOCCF)nc2)cc1]

More preferably, the compound is compounds 87-94.

In a second aspect, the present disclosure provides derivatives of the compounds, which comprise, but are not limited to, pharmaceutically acceptable salts, esters, amide compounds or prodrugs of the compounds represented by the formula (I).

In a third aspect, the present disclosure provides a diagnostic or detecting reagent for neurodegenerative diseases caused by Aβ protein or Tau protein deposition, an active ingredient of which is compounds represented by the formula (I) and/or derivatives thereof.

Preferably, the Aβ protein comprises, but is not limited to, Aβ proteins 1-42.

Further, the diseases comprise, but are not limited to, Alzheimer's disease, frontotemporal lobe degeneration, chronic traumatic encephalopathy, progressive supranuclear palsy, cortical basal ganglia degeneration or Pick's disease.

In a fourth aspect, the present disclosure provides use of compounds represented by the formula (I) and derivatives thereof in the preparation of nuclear medicine imaging agents (such as dual-function imaging agents), optical imaging agents or staining agents.

In a fifth aspect, the present disclosure provides use of compounds represented by the formula (I) and derivatives thereof in the diagnosis of Aβ and Tau-related neurodegenerative diseases.

With the above technical solutions, the present disclosure has at least the following advantages and beneficial effects:

The dihydrazone compounds provided by the present disclosure can be directly used as a fluorescent probe for detecting amyloid plaques and neurofibrillary tangles in vivo or in tissue samples; when staining tissue sections with 100 nM compound solution, the staining is fast, effective and highly specific; when being used for nuclear medicine imaging (positron tomography and single photon computed tomography), it needs to be labelled with a suitable radioisotope. In normal male mice, ¹⁸F-labelled probes can efficiently penetrate the blood-brain barrier and are quickly cleared from the brain. Some probes have no obvious defluorination phenomenon and have good pharmacokinetic properties. Such compounds are particularly suitable for diagnosing neurodegenerative diseases including Alzheimer's disease.

### BRIEF DESCRIPTION OF DRAWINGS

FIG. 1 is schematic diagrams of synthesis processes of the compounds in Examples 1-114 of the present disclosure; reagents and conditions involved are: (a) ethanol, hydrazine hydrate, reflux reaction for 10 minutes at 90° C.

FIG. 2 is fluorescent staining results of the probe 8 in the brain tissue sections of AD patients in Example 8 (A, male, 85 years old, temporal lobe), (B, female, 93 years old, entorhinal cortex).

FIG. 3 is results of autoradiography of [¹⁸F]92 in brain tissue sections of AD patients and normal person in Example 118 of the present disclosure.

FIG. 4 is results of autoradiography of [¹⁸F]91 in brain tissue sections of AD patients in Example 118 of the present disclosure (A, E, G, male, 91 years old, temporal lobe), (B, male, 85 years old, entorhinal cortex), (C, female, 74 years old, temporal lobe), (D, female, 64 years old, temporal lobe), (F, female, 64 years old, temporal lobe), (H, male, 95 years old, temporal lobe), (I, female, 93 years old, temporal lobe); also comprises results of autoradiography of [¹⁸F]91 in bodies of AD transgenic mice and normal mice (J, APP/SP1 mice and normal control mice).

### DETAILED DESCRIPTION OF PREFERRED EMBODIMENTS

The following examples are used to describe the present disclosure, but not to limit the scope of the present disclosure. Unless otherwise specified, the technical means used in the examples are conventional means well known to a person skilled in the art, and raw materials used are all commercially available products.

### Example 1: Synthesis of Compound 1

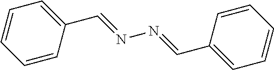 [SMILES: C(=N\N=C\c1ccccc1)/c1ccccc1]

The compound benzaldehyde (212.4 mg, 2.0 mmol) was dissolved in a 50 mL round bottom flask with 10 mL ethanol, and then hydrazine hydrate (58.9 mg, 1.0 mmol) was slowly added to the reaction flask, and the reaction was carried out for 10 minutes at 90° C. under reflux. After the reaction went to completion, cooling until a yellow solid was precipitated. The precipitated product was subjected to suction filtration, and washed with 10 mL of ethanol and petroleum ether. After drying the crystalline product obtained by the suction filtration, 44.0 mg of compound 1 was obtained with a yield of 18.5%. The structure was as follows: ¹H NMR (400 MHz, CDCl₃) δ 8.68 (s, 2H), 7.86 (dd, J=6.5, 3.0 Hz, 4H), 7.56-7.35 (m, 6H).

### Example 2: Synthesis of Compound 2

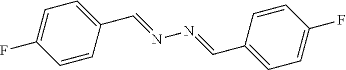 [SMILES: Fc1ccc(/C=N/N=C/c2ccc(F)cc2)cc1]

According to the method for synthesizing compound 1, compound 2 was prepared from p-fluorobenzaldehyde, and 190.0 mg of milky white solid was obtained with a yield of 77.8%. The structure was as follows: ¹H NMR (400 MHz, CDCl₃) δ 8.62 (s, 2H), 7.90-7.74 (m, 4H), 7.21-7.06 (m, 4H).

### Example 3: Synthesis of Compound 3

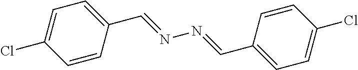 [SMILES: Clc1ccc(/C=N/N=C/c2ccc(Cl)cc2)cc1]

According to the method for synthesizing compound 1, compound 3 was prepared from 4-chlorobenzaldehyde, and 131.0 mg of light yellow crystalline solid was obtained with a yield of 47.3%. The structure was as follows: ¹H NMR (400 MHz, CDCl₃) δ 8.61 (s, 2H), 7.78 (d, J=8.5 Hz, 4H), 7.43 (d, J=8.5 Hz, 4H).

### Example 4: Synthesis of Compound 4

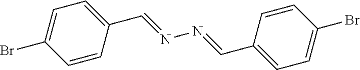 [SMILES: Brc1ccc(/C=N/N=C/c2ccc(Br)cc2)cc1]

According to the method for synthesizing compound 1, compound 4 was prepared from 4-bromobenzaldehyde, and 97.0 mg of light yellow crystalline solid was obtained with a yield of 26.5%. The structure was as follows: ¹H NMR (400 MHz, CDCl₃) δ 8.60 (s, 2H), 7.71 (d, J=8.5 Hz, 4H), 7.59 (d, J=8.4 Hz, 4H).

### Example 5: Synthesis of Compound 5

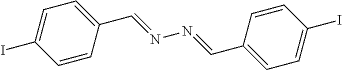 [SMILES: Ic1ccc(/C=N/N=C/c2ccc(I)cc2)cc1]

According to the method for synthesizing compound 1, compound 5 was prepared from 4-iodobenzaldehyde, and 361.0 mg of light yellow crystalline solid was obtained with a yield of 78.5%. The structure was as follows: ¹H NMR (400 MHz, CDCl₃) δ 8.58 (s, 2H), 7.80 (d, J=8.3 Hz, 4H), 7.57 (d, J=8.4 Hz, 4H).

### Example 6: Synthesis of Compound 6

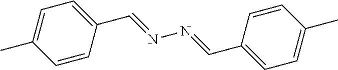

According to the method for synthesizing compound 1, compound 6 was prepared from 4-methylbenzaldehyde, and 68.3 mg of light yellow crystalline solid was obtained with a yield of 28.9%. The structure was as follows: ¹H NMR (400 MHz, CDCl₃) δ 8.65 (s, 2), 7.74 (d, J=8.1 Hz, 4H), 7.38-7.13 (m, 4H), 2.41 (s, 3H).

### Example 7: Synthesis of Compound 7

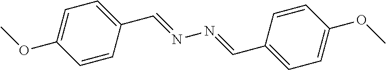

According to the method for synthesizing compound 1, compound 7 was prepared from 4-methoxybenzaldehyde, and 170.4 mg of light yellow crystalline solid was obtained with a yield of 63.5%. The structure was as follows: ¹H NMR (400 MHz, CDCl₃) δ 8.62 (s, 2H), 7.78 (d, J=8.7 Hz, 4H), 6.96 (d, J=8.8 Hz, 4H).

### Example 8: Synthesis of Compound 8

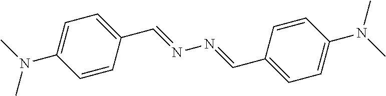

According to the method for synthesizing compound 1, compound 8 (probe 8) was prepared from 4-dimethylaminobenzaldehyde, and 186.0 mg of light yellow crystalline solid was obtained with a yield of 63.2%. The structure was as follows: ¹H NMR (400 MHz, CDCl₃) δ 8.56 (s, 2H), 7.69 (d, J=8.7 Hz, 4H), 6.71 (d, J=8.9 Hz, 4H), 3.02 (s, 12H).

### Example 9: Synthesis of Compound 9

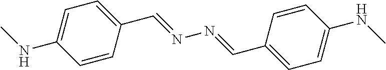

According to the method for synthesizing compound 1, compound 9 was prepared from 4-methylaminobenzaldehyde, and 163.0 mg of light yellow crystalline solid was obtained with a yield of 60.8%. The structure was as follows: ¹H NMR (400 MHz, DMSO-d₆) δ 8.39 (s, 2H), 7.52 (d, J=8.5 Hz, 4H), 6.54 (d, J=8.5 Hz, 4H), 6.28 (q, J=4.8 Hz, 2H), 2.69 (d, J=4.9 Hz, 6H).

### Example 10: Synthesis of Compound 10

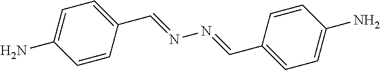 [SMILES: Nc1ccc(/C=N/N=C/c2ccc(N)cc2)cc1]

4-nitrobenzaldehyde (1.5 g, 10.0 mmol) was dissolved in methanol, palladium on carbon (106.4 mg, 1.0 mmol) was added and then hydrazine hydrate (1.8 g, 30.0 mmol) was slowly added dropwise, and the reaction was carried out for 30 minutes at 90° C. under reflux. After the reaction went to completion, the palladium on carbon was removed through suction filtration while hot, and crude product was precipitated in the filtrate, followed by recrystallization with methanol, 103.3 mg of yellow crystalline solid was obtained with a yield of 8.6%. The structure was as follows: ¹H NMR (400 MHz, CDCl₃) δ 8.22-8.11 (m, 4H), 7.72 (s, 2H), 7.69-7.62 (m, 4H), 5.88 (s, 4H).

### Example 11: Synthesis of Compound 11

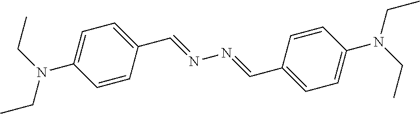 [SMILES: CCN(CC)c1ccc(/C=N/N=C/c2ccc(N(CC)CC)cc2)cc1]

According to the method for synthesizing compound 1, compound 11 was prepared from 4-diethylaminobenzaldehyde, and 48.9 mg of light yellow crystalline solid was obtained with a yield of 27.9%. The structure was as follows: ¹H NMR (400 MHz, CDCl₃) δ 8.53 (s, 2H), 7.65 (d, J=7.8 Hz, 4H), 6.66 (d, J=8.8 Hz, 4H), 3.40 (q, J=7.1 Hz, 8H), 1.19 (t, J=7.1 Hz, 12H).

### Example 12: Synthesis of Compound 12

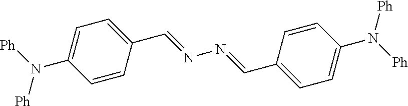 [SMILES: C(=N\N=C\c1ccc(N(c2ccccc2)c2ccccc2)cc1)/c1ccc(N(c2ccccc2)c2ccccc2)cc1]

According to the method for synthesizing compound 1, compound 12 was prepared from 4-diphenylaminobenzaldehyde, and 163.0 mg of yellow crystalline solid was obtained with a yield of 60.1%. The structure was as follows: ¹H NMR (400 MHz, CDCl₃) δ 8.58 (s, 2H), 7.67 (d, J=5.9 Hz, 4H), 7.30 (t, J=7.9 Hz, 8H), 7.15 (d, J=7.5 Hz, 8H), 7.10 (t, J=7.3 Hz, 4H), 7.05 (d, J=8.8 Hz, 4H).

### Example 13: Synthesis of Compound 13

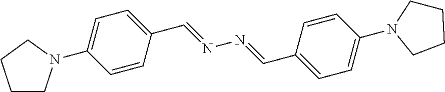 [SMILES: C(=N\N=C\c1ccc(N2CCCC2)cc1)/c1ccc(N2CCCC2)cc1]

According to the method for synthesizing compound 1, compound 13 was prepared from 4-(1-pyrrolidinyl)benzaldehyde, and 30.4 mg of orange crystalline solid was obtained with a yield of 8.8%. The structure was as follows: ¹H NMR (400 MHz, CDCl₃) δ 8.55 (s, 2H), 7.68 (s, 4H), 6.56 (d, J=8.2 Hz, 4H), 3.35 (s, 8H), 2.02 (s, 8H).

### Example 14: Synthesis of Compound 14

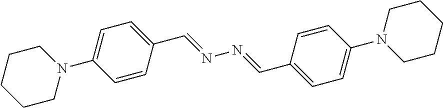 [SMILES: C(=N\N=C\c1ccc(N2CCCCC2)cc1)/c1ccc(N2CCCCC2)cc1]

According to the method for synthesizing compound 1, compound 14 was prepared from 4-(1-piperidinyl)benzaldehyde, and 72.5 mg of yellow crystalline solid was obtained with a yield of 19.4%. The structure was as follows: ¹H NMR (400 MHz, CDCl₃) δ 8.55 (s, 2H), 7.69 (d, J=6.9 Hz, 4H), 6.91 (d, J=8.5 Hz, 4H), 3.29 (d, J=5.5 Hz, 8H), 1.68 (d, J=4.7 Hz, 8H), 1.62 (d, J=4.4 Hz, 4H).

### Example 15: Synthesis of Compound 15

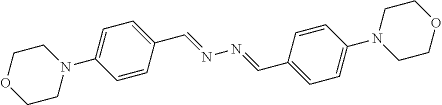 [SMILES: C(=N\N=C\c1ccc(N2CCOCC2)cc1)/c1ccc(N2CCOCC2)cc1]

According to the method for synthesizing compound 1, compound 15 was prepared from 4-(4-morpholinyl)benzaldehyde, and 52.6 mg of light yellow crystalline solid was obtained with a yield of 13.9%. The structure was as follows: ¹H NMR (400 MHz, CDCl₃) δ 8.58 (s, 2H), 7.74 (d, J=7.9 Hz, 4H), 6.91 (d, J=9.0 Hz, 4H), 3.88-3.83 (m, 8H), 3.29-3.25 (m, 8H).

### Example 16: Synthesis of Compound 16

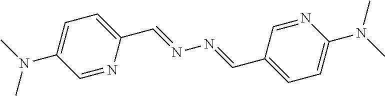

According to the method for synthesizing compound 1, compound 16 was prepared from 6-dimethylaminonicotinaldehyde, 127.0 mg of light yellow crystalline solid was obtained with a yield of 42.8%. The structure was as follows: ¹H NMR (400 MHz, CDCl₃) δ 8.52 (s, 2H), 8.36 (d, J=2.2 Hz, 2H), 8.04 (dd, J=9.0, 2.3 Hz, 2H), 6.56 (d, J=9.0 Hz, 2H), 3.15 (s, 12H).

### Example 17: Synthesis of Compound 17

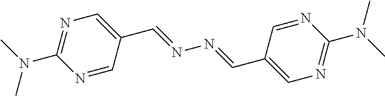

According to the method for synthesizing compound 1, compound 17 was prepared from 2-dimethylaminopyrimidine-5-formaldehyde, 101.3 mg of light yellow crystalline solid was obtained with a yield of 33.9%. The structure was as follows: ¹H NMR (400 MHz, CDCl₃) δ 8.69 (s, 4H), 8.46 (s, 2H), 3.26 (s, 12H).

### Example 18: Synthesis of Compound 18

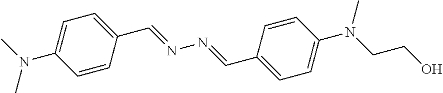 [SMILES: CN(C)c1ccc(/C=N/N=C/c2ccc(N(C)CCO)cc2)cc1]

4-dimethylaminobenzaldehyde (149.6 mg, 1.0 mmol) and N-methyl-N-hydroxyethyl-4-aminobenzaldehyde (179.5 mg, 1.0 mmol) were dissolved in 20 mL ethanol, and hydrazine hydrate (69.3 mg, 1.0 mmol) was added dropwise, the reaction was carried out for 15 minutes at 90° C. under reflux, and ethanol was removed by rotary evaporation under reduced pressure. Column chromatography separation was carried out using petroleum ether and ethyl acetate with a volume ratio of 1:1 as a developing solvent, to obtain 29.3 mg of yellow crystalline solid with a yield of 9.0%. The structure was as follows: ¹H NMR (400 MHz, CDCl₃) δ 8.56 (s, 2H), 7.68 (d, J=7.9 Hz, 4H), 6.77 (d, J=8.4 Hz, 2H), 6.71 (d, J=8.4 Hz, 2H), 3.84 (t, J=5.3 Hz, 2H), 3.56 (t, J=5.7 Hz, 2H), 3.05 (s, 3H), 3.03 (s, 6H).

### Example 19: Synthesis of Compound 19

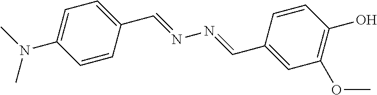

According to the method for synthesizing compound 18, compound 19 was prepared from 4-dimethylaminobenzaldehyde and vanillin, and column chromatography separation was carried out using petroleum ether and ethyl acetate with a volume ratio of 9:1 as a developing solvent, to obtain 102.4 mg of yellow crystalline solid with a yield of 34.4%. The structure was as follows: ¹H NMR (600 MHz, CDCl₃) δ 8.56 (s, 2H), 7.70 (s, 2H), 7.51 (s, 1H), 7.18 (d, J=7.8 Hz, 1H), 6.94 (d, J=8.2 Hz, 1H), 6.71 (d, J=8.4 Hz, 2H), 3.97 (s, 3H), 3.04 (s, 6H).

### Example 20: Synthesis of Compound 20

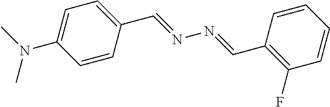 [SMILES: CN(C)c1ccc(/C=N/N=C/c2ccccc2F)cc1]

According to the method for synthesizing compound 18, compound 20 was prepared from 4-dimethylaminobenzaldehyde and 2-fluorobenzaldehyde, and column chromatography separation was carried out using petroleum ether and ethyl acetate with a volume ratio of 10:1 as a developing solvent, to obtain 84.0 mg of yellow crystalline solid with a yield of 31.2%. The structure was as follows: ¹H NMR (400 MHz, CDCl₃) δ 8.91 (s, 1H), 8.58 (s, 1H), 8.09 (td, J=7.6, 1.6 Hz, 1H), 7.72 (d, J=7.8 Hz, 2H), 7.44-7.34 (m, 1H), 7.19 (t, J=7.6 Hz, 1H), 7.12-7.05 (m, 1H), 6.72 (d, J=9.0 Hz, 2H), 3.05 (s, 6H).

### Example 21: Synthesis of Compound 21

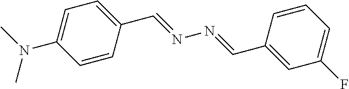 [SMILES: CN(C)c1ccc(/C=N/N=C/c2cccc(F)c2)cc1]

According to the method for synthesizing compound 18, compound 21 was prepared from 4-dimethylaminobenzaldehyde and 3-fluorobenzaldehyde, and column chromatography separation was carried out using petroleum ether and ethyl acetate with a volume ratio of 12:1 as a developing solvent, to obtain 149.8 mg of yellow crystalline solid with a yield of 55.6%. The structure was as follows: ¹H NMR (400 MHz, CDCl₃) δ 8.62 (s, 1H), 8.56 (s, 1H), 7.72 (s, 2H), 7.60-7.45 (m, 2H), 7.38 (td, J=7.9, 5.7 Hz, 1H), 7.11 (td, J=8.5, 2.6 Hz, 1H), 6.72 (d, J=8.9 Hz, 2H), 3.05 (s, 6H).

### Example 22: Synthesis of Compound 22

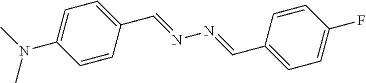 [SMILES: CN(C)c1ccc(/C=N/N=C/c2ccc(F)cc2)cc1]

According to the method for synthesizing compound 18, compound 22 was prepared from 4-dimethylaminobenzaldehyde and 4-fluorobenzaldehyde, and column chromatography separation was carried out using petroleum ether and ethyl acetate with a volume ratio of 15:1 as a developing solvent, to obtain 79.5 mg of yellow crystalline solid with a yield of 29.5%. The structure was as follows: ¹H NMR (400 MHz, CDCl₃) δ 8.62 (s, 1H), 8.57 (s, 1H), 7.83-7.77 (m, 2: H), 7.71 (d, J=8.0 Hz, 2H), 7.10 (m, 2H), 6.72 (d, J=9.0 Hz, 2H), 3.04 (s, 6H).

### Example 23: Synthesis of Compound 23

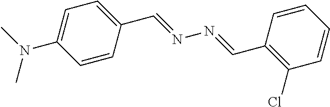 [SMILES: CN(C)c1ccc(/C=N/N=C/c2ccccc2Cl)cc1]

According to the method for synthesizing compound 18, compound 23 was prepared from 4-dimethylaminobenzaldehyde and 2-chlorobenzaldehyde, and column chromatography separation was carried out using petroleum ether and ethyl acetate with a volume ratio of 15:1 as a developing solvent, to obtain 125.3 mg of yellow crystalline solid with a yield of 43.8%. The structure was as follows: ¹H NMR (400 MHz, CDCl₃) δ 9.07 (s, 1H), 8.58 (s, 1H), 8.17 (dd, J=7.3, 2.1 Hz, 1H), 7.73 (d, J=7.6 Hz, 2H), 7.39 (dt, J=8.1, 4.1 Hz, 1H), 7.35-7.27 (m, 2H), 6.72 (d, J=8.9 Hz, 2H), 3.05 (s, 6H).

### Example 24: Synthesis of Compound 24

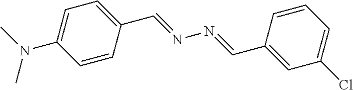 [SMILES: CN(C)c1ccc(/C=N/N=C/c2cccc(Cl)c2)cc1]

According to the method for synthesizing compound 18, compound 24 was prepared from 4-dimethylaminobenzaldehyde and 3-chlorobenzaldehyde, and column chromatography separation was carried out using petroleum ether and ethyl acetate with a volume ratio of 10:1 as a developing solvent, to obtain 130.8 mg of yellow crystalline solid with a yield of 45.8%. The structure was as follows: ¹H NMR (400 MHz, CDCl₃) δ 8.59 (s, 1H), 8.57 (s, 1H), 7.84 (s, 1H), 7.72 (d, J=7.9 Hz, 2H), 7.65 (d, J=7.2 Hz, 1H), 7.44-7.29 (m, 2H), 6.71 (d, J=8.9 Hz, 2H), 3.05 (s, 6H).

### Example 25: Synthesis of Compound 25

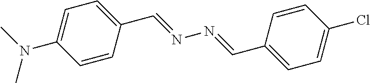 [SMILES: CN(C)c1ccc(/C=N/N=C/c2ccc(Cl)cc2)cc1]

According to the method for synthesizing compound 18, compound 25 was prepared from 4-dimethylaminobenzaldehyde and 4-chlorobenzaldehyde, and column chromatography separation was carried out using petroleum ether and ethyl acetate with a volume ratio of 10:1 as a developing solvent, to obtain 100.8 mg of yellow crystalline solid with a yield of 35.3%. The structure was as follows: ¹H NMR (400 MHz, CDCl₃) δ 8.61 (s, 1H), 8.56 (s, 1H), 7.75 (d, J=8.4 Hz, 4H), 7.39 (d, J=8.3 Hz, 2H), 6.72 (d, J=8.7 Hz, 2H), 3.05 (s, 6H).

### Example 26: Synthesis Compound 26

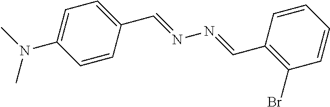 [SMILES: CN(C)c1ccc(/C=N/N=C/c2ccccc2Br)cc1]

According to the method for synthesizing compound 18, compound 26 was prepared from 4-dimethylaminobenzaldehyde and 2-bromobenzaldehyde, and column chromatography separation was carried out using petroleum ether and ethyl acetate with a volume ratio of 8:1 as a developing solvent, to obtain 123.8 mg of yellow crystalline solid with a yield of 37.5%. The structure was as follows: ¹H NMR (400 MHz, CDCl₃) δ 8.59 (s, 1H), 8.17 (d, J=7.7 Hz, 1H), 7.73 (s, 2H), 7.60 (dd, J=8.0, 1.0 Hz, 1H), 7.36 (t, J=7.4 Hz, 1H), 7.26 (s, 2H), 6.73 (d, J=8.8 Hz, 2H), 3.06 (s, 6H).

### Example 27: Synthesis of Compound 27

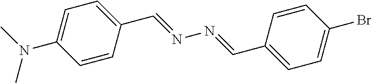 [SMILES: CN(C)c1ccc(/C=N/N=C/c2ccc(Br)cc2)cc1]

According to the method for synthesizing compound 18, compound 27 was prepared from 4-dimethylaminobenzaldehyde and 4-bromobenzaldehyde, and column chromatography separation was carried out using petroleum ether and ethyl acetate with a volume ratio of 10:1 as a developing solvent, to obtain 676.0 mg of yellow crystalline solid with a yield of 40.9%. The structure was as follows: ¹H NMR (400 MHz, CDCl₃) δ 8.58 (s, 1H), 8.55 (s, 1H), 7.72-7.65 (m, 4H), 7.61-7.50 (m, 2H), 6.72 (d, J=9.0 Hz, 2H), 3.05 (s, 6H).

### Example 28: Synthesis of Compound 28

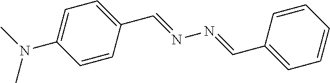 [SMILES: CN(C)c1ccc(/C=N/N=C/c2ccccc2)cc1]

According to the method for synthesizing compound 18, compound 28 was prepared from 4-dimethylaminobenzaldehyde and benzaldehyde, and column chromatography separation was carried out using petroleum ether and ethyl acetate with a volume ratio of 6:1 as a developing solvent, to obtain 101.8 mg of yellow crystalline solid with a yield of 40.5%. The structure was as follows: ¹H NMR (400 MHz, CDCl₃) δ 8.66 (s, 1H), 8.59 (s, 1H), 7.85-7.78 (m, 2H), 7.72 (d, J=6.8 Hz, 2H), 7.45-7.40 (m, 3H), 6.72 (d, J=8.9 Hz, 2H), 3.04 (s, 6H)

### Example 29: Synthesis of Compound 29

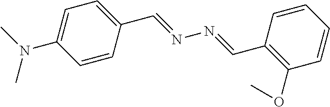 [SMILES: COc1ccccc1/C=N/N=C/c1ccc(N(C)C)cc1]

According to the method for synthesizing compound 18, compound 29 was prepared from 4-dimethylaminobenzaldehyde and 2-methoxybenzaldehyde, and column chromatography separation was carried out using petroleum ether and ethyl acetate with a volume ratio of 5:1 as a developing solvent, to obtain 135.6 mg of yellow crystalline solid with a yield of 48.2%. The structure was as follows: ¹H NMR (400 MHz, CDCl₃) δ 9.07 (s, 1H), 8.56 (s, 1H), 8.05 (d, J=7.1 Hz, 1H), 7.72 (s, 2H), 7.39 (t, J=7.7 Hz, 1H), 7.02-6.95 (m, 1H), 6.92 (d, J=8.4 Hz, 1H), 6.72 (d, J=8.5 Hz, 2H), 3.87 (s, 3H), 3.05 (s, 6H).

### Example 30: Synthesis of Compound 30

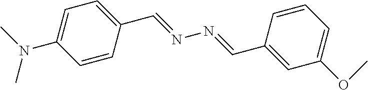

According to the method for synthesizing compound 18, compound 30 was prepared from 4-dimethylaminobenzaldehyde and 3-methoxybenzaldehyde, and column chromatography separation was carried out using petroleum ether and ethyl acetate with a volume ratio of 7:1 as a developing solvent, to obtain 117.6 mg of yellow crystalline solid with a yield of 41.8%. The structure was as follows: ¹H NMR (400 MHz, CDCl₃) δ 8.62 (s, 1H), 8.57 (s, 1H), 7.72 (s, 2H), 7.42 (d, J=2.3 Hz, 1H), 7.33 (dd, J=6.5, 2.4 Hz, 2H), 7.01-6.94 (m, 1H), 6.72 (d, J=8.9 Hz, 2H), 3.86 (s, 3H), 3.05 (s, 6H).

### Example 31: Synthesis of Compound 31

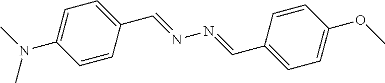

According to the method for synthesizing compound 18, compound 31 was prepared from 4-dimethylaminobenzaldehyde and 4-methoxybenzaldehyde, and column chromatography separation was carried out using petroleum ether and ethyl acetate with a volume ratio of 4:1 as a developing solvent, to obtain 44.1 mg of yellow crystalline solid with a yield of 15.8%. The structure was as follows: ¹H NMR (400 MHz, CDCl₃) δ 8.62 (s, 1H), 8.56 (s, 1H), 7.82-7.76 (m, 2H), 7.75-7.67 (m, 2H), 6.97-6.91 (m, 2H), 6.72 (d, J=9.0 Hz, 2H), 3.85 (s, 3H), 3.04 (s, 6H).

### Example 32: Synthesis of Compound 32

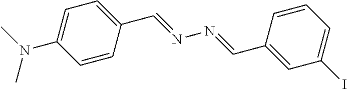 [SMILES: CN(C)c1ccc(/C=N/N=C/c2cccc(I)c2)cc1]

According to the method for synthesizing compound 18, compound 32 was prepared from 4-dimethylaminobenzaldehyde and 3-iodobenzaldehyde, and column chromatography separation was carried out using petroleum ether and ethyl acetate with a volume ratio of 12:1 as a developing solvent, to obtain 163.9 mg of yellow crystalline solid with a yield of 43.4%. The structure was as follows: ¹H NMR (600 MHz, CDCl₃) δ 8.57 (s, 1H), 8.53 (s, 1H), 8.20 (s, 1H), 7.73 (dd, J=7.7, 6.0 Hz, 4H), 7.15 (t, J=7.7 Hz, 1H), 6.71 (d, J=8.8 Hz, 2H), 3.05 (s, 6H).

### Example 33: Synthesis of Compound 33

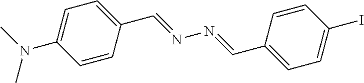 [SMILES: CN(C)c1ccc(/C=N/N=C/c2ccc(I)cc2)cc1]

According to the method for synthesizing compound 18, compound 33 was prepared from 4-dimethylaminobenzaldehyde and 4-iodobenzaldehyde, and column chromatography separation was carried out using petroleum ether and ethyl acetate with a volume ratio of 10:1 as a developing solvent, to obtain 38.3 mg of yellow crystalline solid with a yield of 10.2%. The structure was as follows: ¹H NMR (600 MHz, CDCl₃) δ 8.56 (s, 2H), 7.76 (d, J=8.2 Hz, 2H), 7.71 (d, J=5.6 Hz, 2H), 7.53 (d, J=8.3 Hz, 2H), 6.71 (d, J=8.8 Hz, 2H), 3.05 (s, 6H).

### Example 34: Synthesis of Compound 34

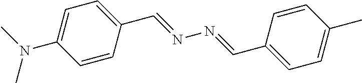

According to the method for synthesizing compound 18, compound 34 was prepared from 4-dimethylaminobenzaldehyde and 4-methylbenzaldehyde, and column chromatography separation was carried out using petroleum ether and ethyl acetate with a volume ratio of 4:1 as a developing solvent, to obtain 98.9 mg of yellow crystalline solid with a yield of 37.3%. The structure was as follows: ¹H NMR (400 MHz, CDCl₃) δ 8.63 (s, 1H), 8.57 (s, 1H), 7.71 (d, J=8.1 Hz, 4H), 7.23 (d, J=8.2 Hz, 2H), 6.72 (d, J=9.0 Hz, 2H), 3.04 (s, 6H), 2.39 (s, 3H).

### Example 35: Synthesis of Compound 35

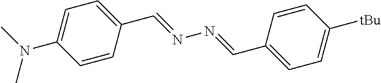 [SMILES: CN(C)c1ccc(/C=N/N=C/c2ccc(C(C)(C)C)cc2)cc1]

According to the method for synthesizing compound 18, compound 35 was prepared from 4-dimethylaminobenzaldehyde and 4-tert-butylbenzaldehyde, and column chromatography separation was carried out using petroleum ether and ethyl acetate with a volume ratio of 9:1 as a developing solvent, to obtain 114.3 mg of yellow crystalline solid with a yield of 46.9%. The structure was as follows: ¹H NMR (400 MHz, CDCl₃) δ 8.63 (s, 1H), 8.57 (s, 1H), δ 7.78-7.66 (m, 4H), 7.47-7.42 (m, 2H), 6.71 (t, J=6.6 Hz, 2H), 3.05 (s, 6H), 1.33 (s, 9H).

### Example 36: Synthesis of Compound 36

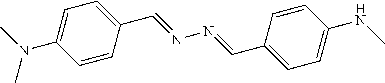

According to the method for synthesizing compound 18, compound 36 was prepared from 4-dimethylaminobenzaldehyde and 4-methylaminobenzaldehyde, and column chromatography separation was carried out using petroleum ether and ethyl acetate with a volume ratio of 5:1 as a developing solvent, to obtain 74.3 mg of yellow crystalline solid with a yield of 26.5%. The structure was as follows: ¹H NMR (400 MHz, CDCl₃) δ 8.56 (s, 2H), 7.68 (dd, J=14.3, 8.3 Hz, 4H), 6.71 (d, J=9.1 Hz, 2H), 6.59 (t, J=8.7 Hz, 2H), 4.10 (s, 1H), 3.03 (s, 6H), 2.88 (s, 3H).

### Example 37: Synthesis of Compound 37

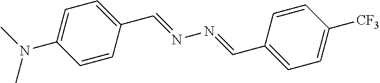 [SMILES: CN(C)c1ccc(/C=N/N=C/c2ccc(C(F)(F)F)cc2)cc1]

According to the method for synthesizing compound 18, compound 37 was prepared from 4-dimethylaminobenzaldehyde and 4-trifluoromethylbenzaldehyde, and column chromatography separation was carried out using petroleum ether and ethyl acetate with a volume ratio of 5:1 as a developing solvent, to obtain 142.0 mg of yellow crystalline solid with a yield of 44.5%. The structure was as follows: ¹H NMR (400 MHz, CDCl₃) δ 8.68 (s, 1H), 8.61 (s, 1H), 7.93 (d, J=8.1 Hz, 2H), 7.74 (d, J=8.4 Hz, 2H), 7.68 (d, J=8.2 Hz, 2H), 6.73 (d, J=9.0 Hz, 2H), 3.06 (s, 6H).

### Example 38: Synthesis of Compound 38

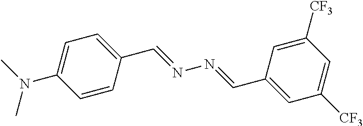 [SMILES: CN(C)c1ccc(/C=N/N=C/c2cc(C(F)(F)F)cc(C(F)(F)F)c2)cc1]

According to the method for synthesizing compound 18, compound 38 was prepared from 4-dimethylaminobenzaldehyde and 3,5-bistrifluoromethylbenzaldehyde, and column chromatography separation was carried out using petroleum ether and ethyl acetate with a volume ratio of 11:1 as a developing solvent, to obtain 113.6 mg of yellow crystalline solid with a yield of 29.3%. The structure was as follows: ¹H NMR (400 MHz, CDCl₃) δ 8.68 (s, 1H), 8.61 (s, 1H), 8.24 (s, 2H), 7.89 (s, 1H), 7.74 (d, J=8.0 Hz, 2H), 6.72 (d, J=8.9 Hz, 2H), 3.06 (s, 6H).

### Example 39: Synthesis of Compound 39

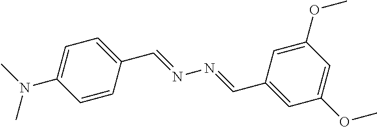

According to the method for synthesizing compound 18, compound 39 was prepared from 4-dimethylaminobenzaldehyde and 3,5-dimethoxybenzaldehyde, and column chromatography separation was carried out using dichloromethane, petroleum ether and ethyl acetate with a volume ratio of 15:10:1 as a developing solvent, to obtain 82.6 mg of yellow crystalline solid with a yield of 26.5%. The structure was as follows: ¹H NMR (400 MHz, CDCl₃) δ 8.58 (s, 2H), 7.71 (s, 2H), 6.97 (d, J=8.7 Hz, 2H), 6.72 (d, J=8.7 Hz, 2H), 6.53 (t, J=2.2 Hz, 1H), 3.83 (s, 6H), 3.04 (s, 6H).

### Example 40: Synthesis of Compound 40

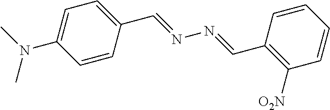 [SMILES: CN(C)c1ccc(/C=N/N=C/c2ccccc2[N+](=O)[O-])cc1]

According to the method for synthesizing compound 17, compound 40 was prepared from 4-dimethylaminobenzaldehyde and 2-nitrobenzaldehyde, and column chromatography separation was carried out using dichloromethane, petroleum ether and ethyl acetate with a volume ratio of 20:20:1 as a developing solvent, to obtain 105.9 mg of yellow crystalline solid with a yield of 35.7%. The structure was as follows: ¹H NMR (400 MHz, CDCl₃) δ 9.11 (s, 1H), 8.56 (s, 1H), 8.23 (d, J=7.8 Hz, 1H), 8.02 (dd, J=8.2, 1.1 Hz, 1H), 7.75 (s, 2H), 7.66 (t, J=7.5 Hz, 1H), 7.59-7.52 (m, 1H), 6.72 (d, J=8.9 Hz, 2H), 3.06 (s, 6H).

### Example 41: Synthesis of Compound 41

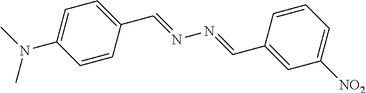 [SMILES: CN(C)c1ccc(/C=N/N=C/c2cccc([N+](=O)[O-])c2)cc1]

According to the method for synthesizing compound 17, compound 41 was prepared from 4-dimethylaminobenzaldehyde and 3-nitrobenzaldehyde, and column chromatography separation was carried out using dichloromethane, petroleum ether and ethyl acetate with a volume ratio of 10:15:1 as a developing solvent, to obtain 153.8 mg of yellow crystalline solid with a yield of 51.9%. The structure was as follows: ¹H NMR (400 MHz, CDCl₃) δ 8.71 (s, 1H), 8.67 (s, 1H), 8.62 (s, 1H), 8.32-8.23 (m, 1H), 8.13 (d, J=7.7 Hz, 1H), 7.75 (d, J=6.6 Hz, 2H), 7.61 (t, J=8.0 Hz, 1H), 6.73 (d, J=8.5 Hz, 2H), 3.07 (s, 6H)

### Example 42: Synthesis of Compound 42

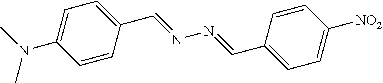 [SMILES: CN(C)c1ccc(/C=N/N=C/c2ccc([N+](=O)[O-])cc2)cc1]

According to the method for synthesizing compound 17, compound 41 was prepared from 4-dimethylaminobenzaldehyde and 4-nitrobenzaldehyde, and column chromatography separation was carried out using dichloromethane, petroleum ether and ethyl acetate with a volume ratio of 10:15:1 as a developing solvent, to obtain 29.5 mg of yellow crystalline solid with a yield of 10.0%. The structure was as follows: ¹H NMR (400 MHz, CDCl₃) δ 8.70 (s, 1H), 8.60 (s, 1H), 8.30-8.22 (m, 2H), 7.99-7.94 (m, 2H), 7.74 (d, J=6.1 Hz, 2H), 6.72 (d, J=9.1 Hz, 2H), 3.07 (s, 6H).

### Example 43: Synthesis of Compound 43

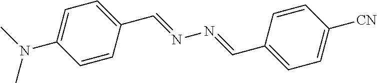 [SMILES: CN(C)c1ccc(/C=N/N=C/c2ccc(C#N)cc2)cc1]

According to the method for synthesizing compound 17, compound 43 was prepared from 4-dimethylaminobenzaldehyde and 4-cyanobenzaldehyde, and column chromatography separation was carried out using dichloromethane, petroleum ether and ethyl acetate with a volume ratio of 10:20:1 as a developing solvent, to obtain 118.9 mg of yellow crystalline solid with a yield of 43.0%. The structure was as follows: ¹H NMR (600 MHz, CDCl₃) δ 8.63 (s, 1H), 8.57 (s, 1H), 7.89 (t, J=11.6 Hz, 2H), 7.79-7.61 (m, 4H), 6.72 (d, J=8.6 Hz, 2H), 3.06 (s, 6H).

### Example 44: Synthesis of Compound 44

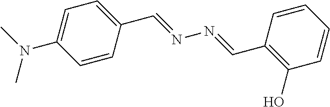 [SMILES: CN(C)c1ccc(/C=N/N=C/c2ccccc2O)cc1]

According to the method for synthesizing compound 18, compound 44 was prepared from 4-dimethylaminobenzaldehyde and 2-hydroxybenzaldehyde, and column chromatography separation was carried out using petroleum ether and ethyl acetate with a volume ratio of 5:1 as a developing solvent, to obtain 58.9 mg of yellow crystalline solid with a yield of 22.0%. The structure was as follows: ¹H NMR (600 MHz, CDCl₃) δ 8.71 (s, 1H), 8.50 (s, 1H), 7.31 (t, J=7.1 Hz, 2H), 7.00 (d, J=8.6 Hz, 2H), 6.91 (t, J=7.5 Hz, 2H), 6.72 (d, J=8.5 Hz, 2H), 3.06 (s, 6H).

### Example 45: Synthesis of Compound 45

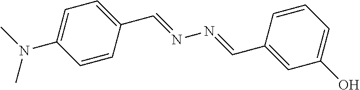 [SMILES: CN(C)c1ccc(/C=N/N=C/c2cccc(O)c2)cc1]

According to the method for synthesizing compound 18, compound 45 was prepared from 4-dimethylaminobenzaldehyde and 3-hydroxybenzaldehyde, and column chromatography separation was carried out using dichloromethane, petroleum ether and ethyl acetate with a volume ratio of 10:20:1 as a developing solvent, to obtain 43.5 mg of yellow crystalline solid with a yield of 16.3%. The structure was as follows: ¹H NMR (600 MHz, CDCl₃) δ 8.60 (s, 1H), 8.55 (s, 1H), 7.72 (s, 2H), 7.37-7.26 (m, 3H), 6.91 (dd, J=4.5, 3.3 Hz, 1H), 6.71 (d, J=8.7 Hz, 2H), 3.05 (s, 6H).

### Example 46: Synthesis of Compound 46

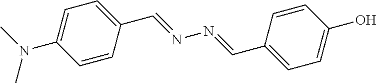 [SMILES: CN(C)c1ccc(/C=N/N=C/c2ccc(O)cc2)cc1]

According to the method for synthesizing compound 18, compound 46 was prepared from 4-dimethylaminobenzaldehyde and 4-hydroxybenzaldehyde, and column chromatography separation was carried out using dichloromethane, petroleum ether and ethyl acetate with a volume ratio of 10:15:1 as a developing solvent, to obtain 12.0 mg of yellow crystalline solid with a yield of 4.5%. The structure was as follows: ¹H NMR (600 MHz, DMSO-d6) δ 8.48 (d, J=13.5 Hz, 2H), 7.62 (t, J=7.6 Hz, 4H), 6.81 (d, J=7.5 Hz, 2H), 6.73 (d, J=8.1 Hz, 2H), 2.96 (s, 6H).

### Example 47: Synthesis of Compound 47

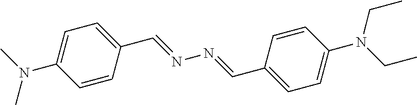 [SMILES: CCN(CC)c1ccc(/C=N/N=C/c2ccc(N(C)C)cc2)cc1]

According to the method for synthesizing compound 18, compound 47 was prepared from 4-dimethylaminobenzaldehyde and 4-diethylaminobenzaldehyde, and column chromatography separation was carried out using petroleum ether and ethyl acetate with a volume ratio of 9:1 as a developing solvent, to obtain 1.0 mg of orange crystalline solid with a yield of 0.3%. The structure was as follows: ¹H NMR (400 MHz, CDCl₃) δ 8.55 (s, 2H), 7.69 (s, 4H), 6.70 (dd, J=15.3, 8.7 Hz, 4H), 3.42 (q, J=7.0 Hz, 4H), 3.04 (s, 6H), 1.20 (s, 6H).

### Example 48: Synthesis of Compound 48

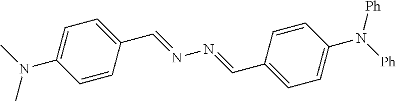 [SMILES: CN(C)c1ccc(/C=N/N=C/c2ccc(N(c3ccccc3)c3ccccc3)cc2)cc1]

According to the method for synthesizing compound 18, compound 48 was prepared from 4-dimethylaminobenzaldehyde and 4-diphenylaminobenzaldehyde, and column chromatography separation was carried out using petroleum ether and ethyl acetate with a volume ratio of 5:1 as a developing solvent, to obtain 45.3 mg of orange crystalline solid with a yield of 10.8%. The structure was as follows: ¹H NMR (400 MHz, CDCl₃) δ 8.59 (s, 1H), 8.57 (s, 1H), 7.71 (d, J=8.4 Hz, 2H), 7.66 (d, J=8.7 Hz, 2H), 7.28 (dd, J=14.3, 6.8 Hz, 4H), 7.16-7.12 (m, 4H), 7.08 (dd, J=14.7, 8.0 Hz, 4H), 6.72 (d, J=9.0 Hz, 2H), 3.05 (s, 6H).

### Example 49: Synthesis of Compound 49

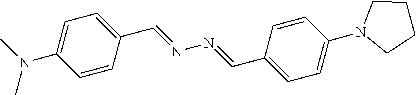 [SMILES: CN(C)c1ccc(/C=N/N=C/c2ccc(N3CCCC3)cc2)cc1]

According to the method for synthesizing compound 18, compound 49 was prepared from 4-dimethylaminobenzaldehyde and 4-(1-pyrrolidinyl)benzaldehyde, and column chromatography separation was carried out using dichloromethane, petroleum ether and ethyl acetate with a volume ratio of 20:5:1 as a developing solvent, to obtain 46.6 mg of yellow crystalline solid with a yield of 14.5%. The structure was as follows: ¹H NMR (400 MHz, CDCl₃) δ 8.56 (s, 2H), 7.69 (d, J=7.0 Hz, 4H), 6.71 (d, J=8.7 Hz, 2H), 6.56 (d, J=8.6 Hz, 2H), 3.35 (t, J=6.3 Hz, 4H), 3.03 (s, 6H), 2.02 (t, J=3.0 Hz, 4H).

### Example 50: Synthesis of Compound 50

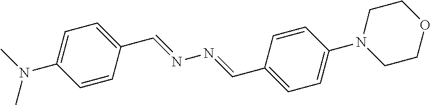 [SMILES: CN(C)c1ccc(/C=N/N=C/c2ccc(N3CCOCC3)cc2)cc1]

According to the method for synthesizing compound 18, compound 50 was prepared from 4-dimethylaminobenzaldehyde and 4-(4-morpholinyl) benzaldehyde, and column chromatography separation was carried out using dichloromethane, petroleum ether and ethyl acetate with a volume ratio of 10:5:1 as a developing solvent, to obtain 80.2 mg of yellow crystalline solid with a yield of 23.8%. The structure was as follows: ¹H NMR (400 MHz, CDCl₃) δ 8.58 (s, 1H), 8.56 (s, 1H), 7.72 (d, J=8.8 Hz, 4H), 6.91 (d, J=8.9 Hz, 2H), 6.71 (d, J=9.0 Hz, 2H), 3.90-3.82 (m, 4H), 3.29-3.23 (m, 4H), 3.03 (s, 6H).

### Example 51: Synthesis of Compound 51

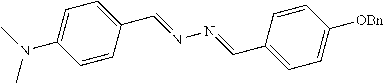 [SMILES: CN(C)c1ccc(/C=N/N=C/c2ccc(OCc3ccccc3)cc2)cc1]

According to the method for synthesizing compound 18, compound 51 was prepared from 4-dimethylaminobenzaldehyde and 4-benzyloxybenzaldehyde, and column chromatography separation was carried out using petroleum ether and ethyl acetate with a volume ratio of 5:1 as a developing solvent, to obtain 136.4 mg of light yellow crystalline solid with a yield of 38.2%. The structure was as follows: ¹H NMR (400 MHz, CDCl₃) δ 8.62 (s, 1H), 8.58 (s, 1H), 7.77 (d, J=8.7 Hz, 2H), 7.71 (d, J=7.9 Hz, 2H), 7.50-7.31 (m, 5H), 7.02 (t, J=8.7 Hz, 2H), 6.72 (d, J=9.0 Hz, 2H), 5.12 (s, 2H), 3.05 (s, 6H).

### Example 52: Synthesis of Compound 52

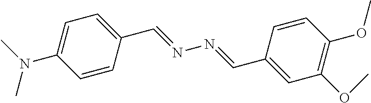

According to the method for synthesizing compound 18, compound 52 was prepared from 4-dimethylaminobenzaldehyde and 3.4-dimethoxybenzaldehyde, and column chromatography separation was carried out using dichloromethane, petroleum ether and ethyl acetate with a volume ratio of 10:5:1 as a developing solvent, to obtain 64.2 mg of light yellow crystalline solid with a yield of 20.6%. The structure was as follows: ¹H NMR (400 MHz, CDCl₃) δ 8.59 (s, 2H), 7.72 (d, J=8.0 Hz, 2H), 7.53 (d, J=1.6 Hz, 1H), 7.23 (dd, J=8.2, 1.7 Hz, 1H), 6.90 (d, J=8.3 Hz, 1H), 6.72 (d, J=9.0 Hz, 2H), 3.97 (s, 3H), 3.93 (s, 3H), 3.04 (s, 6H).

### Example 53: Synthesis of Compound 53

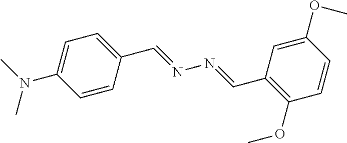

According to the method for synthesizing compound 18, compound 53 was prepared from 4-dimethylaminobenzaldehyde and 2.5-dimethoxybenzaldehyde, and column chromatography separation was carried out using dichloromethane, petroleum ether and ethyl acetate with a volume ratio of 10:20:1 as a developing solvent, to obtain 98.0 mg of yellow crystalline solid with a yield of 31.5%. The structure was as follows: ¹H NMR (400 MHz, CDCl₃) δ 9.02 (s, 1H), 8.58 (s, 1H), 7.71 (d, J=7.3 Hz, 2H), 7.62 (d, J=2.8 Hz, 1H), 6.96 (dd, J=9.0, 3.2 Hz, 1H), 6.87 (d, J=9.0 Hz, 1H), 6.72 (d, J=8.9 Hz, 2H), 3.84 (s, J=2.6 Hz, 6H), 3.04 (s, 6H).

### Example 54: Synthesis of Compound 54

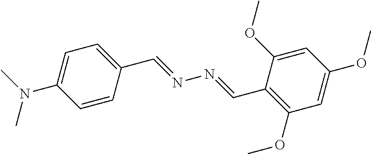

According to the method for synthesizing compound 18, compound 54 was prepared from 4-dimethylaminobenzaldehyde and 2,4,6-trimethoxybenzaldehyde, and column chromatography separation was carried out using petroleum ether and ethyl acetate with a volume ratio of 9:1 as a developing solvent, to obtain 116.4 mg of yellow crystalline solid with a yield of 34.1%. The structure was as follows: ¹H NMR (400 MHz, CDCl₃) δ 9.07 (s, 1H), 8.67 (s, 1H), 7.70 (d, J=8.7 Hz, 2H), 6.71 (d, J=9.0 Hz, 2H), 6.15 (s, 2H), 3.90 (s, 6H), 3.86 (s, 3H), 3.03 (s, 6H).

### Example 55: Synthesis of Compound 55

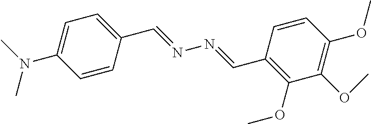

According to the method for synthesizing compound 18, compound 55 was prepared from 4-dimethylaminobenzaldehyde and 2,3,4-trimethoxybenzaldehyde, and column chromatography separation was carried out using dichloromethane, petroleum ether and ethyl acetate with a volume ratio of 10:10:1 as a developing solvent, to obtain 125.7 mg of yellow crystalline solid with a yield of 36.8%. The structure was as follows: ¹H NMR (400 MHz, CDCl₃) δ 8.91 (s, 1H), 8.58 (s, 1H), 7.82 (d, J=8.8 Hz, 1H), 7.71 (d, J=8.3 Hz, 2H), 6.73 (t, J=8.8 Hz, 3H), 3.95 (s, 3H), 3.90 (d, J=6.9 Hz, 6H), 3.04 (s, 6H).

### Example 56: Synthesis of Compound 56

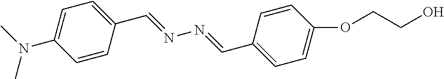 [SMILES: CN(C)c1ccc(/C=N/N=C/c2ccc(OCCO)cc2)cc1]

According to the method for synthesizing compound 18, compound 56 was prepared from 4-dimethylaminobenzaldehyde and 4-(2-hydroxyethoxy)benzaldehyde, and column chromatography separation was carried out using dichloromethane and ethyl acetate with a volume ratio of 1:1 as a developing solvent, to obtain 62.5 mg of yellow crystalline solid with a yield of 20.1%. The structure was as follows: ¹H NMR (400 MHz, CDCl₃) δ 8.61 (s, 1H), 8.56 (s, 1H), 7.79-7.73 (m, 2H), 7.71 (d, J=7.5 Hz, 2H), 6.98-6.93 (m, 2H), 6.71 (d, J=9.0 Hz, 2H), 4.14-4.10 (m, 2H), 3.98 (dd, J=9.4, 5.0 Hz, 2H), 3.04 (s, 6H).

### Example 57: Synthesis of Compound 57

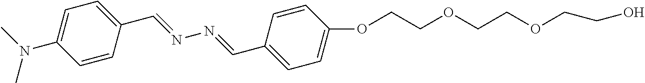 [SMILES: CN(C)c1ccc(/C=N/N=C/c2ccc(OCCOCCOCCO)cc2)cc1]

According to the method for synthesizing compound 18, compound 57 was prepared from 4-dimethylaminobenzaldehyde 4-{2-[2-(2-hydroxyethoxy)ethoxy]ethoxy}benzaldehyde, and column chromatography separation was carried out using dichloromethane and ethyl acetate with a volume ratio of 1:4 as a developing solvent, to obtain 57.3 mg of light yellow crystalline solid with a yield of 14.3%. The structure was as follows: ¹H NMR (400 MHz, CDCl₃) δ 8.60 (s, 1H), 8.56 (s, 1H), 7.76-7.72 (m, 2H), 7.70 (d, J=6.8 Hz, 2H), 6.95 (d, J=9.0 Hz, 2H), 6.71 (d, J=9.0 Hz, 2H), 4.20-4.16 (m, 2H), 3.88 (dd, J=5.4, 4.2 Hz, 2H), 3.75-3.68 (m, 6H), 3.63-3.59 (m, 2H), 3.04 (s, 6H).

### Example 58: Synthesis of Compound 58

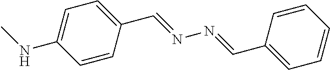 [SMILES: CNc1ccc(/C=N/N=C/c2ccccc2)cc1]

According to the method for synthesizing compound 18, compound 58 was prepared from 4-methylaminobenzaldehyde and benzaldehyde, and column chromatography separation was carried out using petroleum ether and ethyl acetate with a volume ratio of 9:1 as a developing solvent, to obtain 22.8 mg of light yellow crystalline solid with a yield of 9.6%. The structure was as follows: ¹H NMR (400 MHz, CDCl₃) δ 8.67 (s, 1H), 8.58 (s, 1H), 7.82 (dd, J=6.3, 3.1 Hz, 2H), 7.70 (d, J=7.9 Hz, 2H), 7.49-7.39 (m, 3H), 6.63 (d, J=8.6 Hz, 2H), 4.17 (s, 1H), 2.90 (s, 3H).

### Example 59: Synthesis of Compound 59

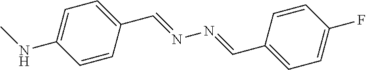 [SMILES: CNc1ccc(/C=N/N=C/c2ccc(F)cc2)cc1]

According to the method for synthesizing compound 18, compound 59 was prepared from 4-methylaminobenzaldehyde and 4-fluorobenzaldehyde, and column chromatography separation was carried out using dichloromethane, petroleum ether and ethyl acetate with a volume ratio of 15:5:1 as a developing solvent, to obtain 25.6 mg of light yellow crystalline solid with a yield of 9.6%. The structure was as follows: ¹H NMR (400 MHz, CDCl₃) δ 8.62 (s, 1H), 8.56 (s, 1H), 7.81 (dd, J=8.7, 5.5 Hz, 2H), 7.69 (d, J=8.0 Hz, 2H), 7.12 (t, J=8.7 Hz, 2H), 6.62 (d, J=8.7 Hz, 2H), 2.90 (s, 3H).

### Example 60: Synthesis of Compound 60

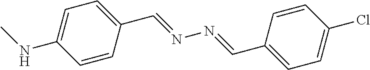 [SMILES: CNc1ccc(/C=N/N=C/c2ccc(Cl)cc2)cc1]

According to the method for synthesizing compound 18, compound 60 was prepared from 4-methylaminobenzaldehyde and 4-chlorobenzaldehyde, and column chromatography separation was carried out using dichloromethane, petroleum ether and ethyl acetate with a volume ratio of 15:10:1 as a developing solvent, to obtain 18.1 mg of light yellow crystalline solid with a yield of 6.7%. The structure was as follows: ¹H NMR (400 MHz, CDCl₃) δ 8.61 (s, 1H), 8.56 (s, 1H), 7.80-7.75 (m, 2H), 7.70 (d, J=7.5 Hz, 2H), 7.42 (dd, J=11.1, 8.5 Hz, 2H), 6.63 (d, J=8.8 Hz, 2H), 2.91 (s, 3H).

### Example 61: Synthesis of Compound 61

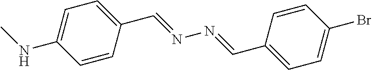 [SMILES: CNc1ccc(/C=N/N=C/c2ccc(Br)cc2)cc1]

According to the method for synthesizing compound 18, compound 61 was prepared from 4-methylaminobenzaldehyde and 4-bromobenzaldehyde, and column chromatography separation was carried out using petroleum ether and ethyl acetate with a volume ratio of 5:1 as a developing solvent, to obtain 1.8 mg of light yellow crystalline solid with a yield of 0.6%. The structure was as follows: ¹H NMR (400 MHz, CDCl₃) δ 8.63 (s, 1H), 8.55 (s, 1H), 7.69 (d, J=8.5 Hz, 4H), 7.56 (d, J=8.5 Hz, 2H), 6.63 (d, J=8.8 Hz, 2H), 2.91 (s, 3H).

### Example 62: Synthesis of Compound 62

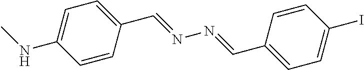 [SMILES: CNc1ccc(/C=N/N=C/c2ccc(I)cc2)cc1]

According to the method for synthesizing compound 18, compound 62 was prepared from 4-methylaminobenzaldehyde and 4-iodobenzaldehyde, and column chromatography separation was carried out using petroleum ether and ethyl acetate with a volume ratio of 4:1 as a developing solvent, to obtain 22.0 mg of yellow crystalline solid with a yield of 6.0%. The structure was as follows: ¹H NMR (400 MHz, CDCl₃) δ 8.56 (s, 1H), 8.56-8.54 (m, 1H), 7.76 (d, J=8.4 Hz, 2H), 7.68 (d, J=7.8 Hz, 2H), 7.53 (d, J=8.4 Hz, 2H), 6.61 (d, J=8.8 Hz, 2H), 2.90 (s, 3H).

### Example 63: Synthesis of Compound 63

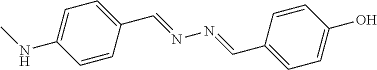 [SMILES: CNc1ccc(/C=N/N=C/c2ccc(O)cc2)cc1]

According to the method for synthesizing compound 18, compound 63 was prepared from 4-methylaminobenzaldehyde and 4-hydroxybenzaldehyde, and column chromatography separation was carried out using dichloromethane, petroleum ether and ethyl acetate with a volume ratio of 15:5:2 as a developing solvent, to obtain 24.9 mg of orange-red crystalline solid with a yield of 9.8%. The structure was as follows: ¹H NMR (400 MHz, DMSO-d₆) δ 8.49 (s, 1H), 8.45 (s, 1H), 7.64 (d, J=8.6 Hz, 2H), 7.56 (d, J=8.7 Hz, 2H), 6.82 (d, J=8.6 Hz, 2H), 6.57 (d, J=8.7 Hz, 2H), 6.38-6.34 (m, 1H), 2.71 (d, J=4.9 Hz, 3H).

### Example 64: Synthesis of Compound 64

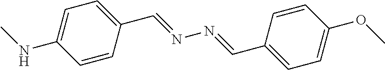

According to the method for synthesizing compound 18, compound 64 was prepared from 4-methylaminobenzaldehyde and 4-methoxybenzaldehyde, and column chromatography separation was carried out using dichloromethane, petroleum ether and ethyl acetate with a volume ratio of 5:5:1 as a developing solvent, to obtain 42.6 mg of yellow crystalline solid with a yield of 15.9%. The structure was as follows: ¹H NMR (400 MHz, CDCl₃) δ 8.62 (s, 1H), 8.56 (s, 1H), 7.82-7.73 (m, 2H), 7.68 (d, J=8.3 Hz, 2H), 6.95 (d, J=8.8 Hz, 2H), 6.62 (d, J=8.8 Hz, 2H), 3.86 (s, 3H), 2.90 (s, 3H).

### Example 65: Synthesis of Compound 56

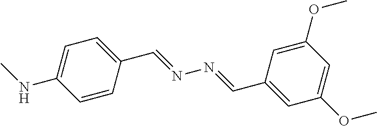

According to the method for synthesizing compound 18, compound 65 was prepared from 4-methylaminobenzaldehyde and 3.5-dimethoxybenzaldehyde, and column chromatography separation was carried out using petroleum ether and ethyl acetate with a volume ratio of 5:1 as a developing solvent, to obtain 43.4 mg of light yellow crystalline solid with a yield of 14.6%. The structure was as follows: ¹H NMR (400 MHz, CDCl₃) δ 9.04 (s, 1H), 8.57 (s, 1H), 7.69 (d, J=8.1 Hz, 2H), 7.64 (dd, J=9.1, 3.1 Hz, 1H), 7.01-6.94 (m, 1H), 6.88 (dd, J=9.0, 4.5 Hz, 1H), 6.62 (d, J=8.6 Hz, 2H), 3.84 (s, 6H), 2.90 (s, 3H).

### Example 66: Synthesis of Compound 66

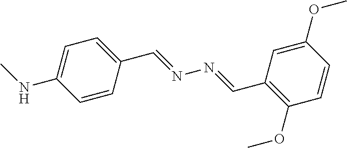

According to the method for synthesizing compound 18, compound 66 was prepared from 4-methylaminobenzaldehyde and 2.5-dimethoxybenzaldehyde, and column chromatography separation was carried out using petroleum ether and ethyl acetate with a volume ratio of 4:1 as a developing solvent, to obtain 29.5 mg of yellow crystalline solid with a yield of 9.9%. The structure was as follows: ¹H NMR (400 MHz, CDCl₃) δ 9.08 (s, 1H), 8.56 (s, 1H), 7.67-7.72 (m, 3H), 6.98 (dd, J=9.0, 3.1 Hz, 1H), 6.88 (d, J=9.0 Hz, 1H), 6.63 (d, J=8.4 Hz, 2H), 3.84 (s, 3H), 3.83 (s, 3H), 2.91 (s, 3H).

### Example 67: Synthesis of Compound 67

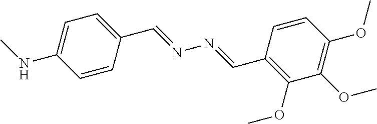

According to the method for synthesizing compound 18, compound 67 was prepared from 4-methylaminobenzaldehyde and 2,3,4-trimethoxybenzaldehyde, and column chromatography separation was carried out using dichloromethane, petroleum ether and ethyl acetate with a volume ratio of 5:10:2 as a developing solvent, to obtain 53.0 mg of yellow crystalline solid with a yield of 16.2%. The structure was as follows: ¹H NMR (400 MHz, CDCl₃) δ 8.91 (s, 1H), 8.56 (s, 1H), 7.82 (d, J=8.8 Hz, 1H), 7.69 (d, J=7.7 Hz, 2H), 6.74 (d, J=8.9 Hz, 1H), 6.62 (d, J=8.5 Hz, 2H), 4.12 (s, 1H), 3.95 (s, 3H), 3.90 (d, J=7.6 Hz, 6H), 2.90 (s, 3H).

### Example 68: Synthesis of Compound 68

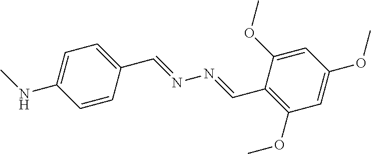

According to the method for synthesizing compound 18, compound 68 was prepared from 4-methylaminobenzaldehyde and 2,4,6-trimethoxybenzaldehyde, and column chromatography separation was carried out using dichloromethane, petroleum ether and ethyl acetate with a volume ratio of 5:10:2 as a developing solvent, to obtain 57.8 mg of yellow crystalline solid with a yield of 17.6%. The structure was as follows: ¹H NMR (400 MHz, CDCl₃) δ 9.06 (s, 1H), 8.67 (s, 1H), 7.67 (d, J=8.5 Hz, 2H), 6.60 (d, J=8.7 Hz, 2H), 6.15 (s, 2H), 4.08 (s, 1H), 3.90 (s, 6H), 3.86 (s, 3H), 2.88 (s, 3H).

### Example 69: Synthesis of Compound 69

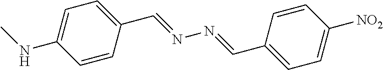 [SMILES: CNc1ccc(/C=N/N=C/c2ccc([N+](=O)[O-])cc2)cc1]

According to the method for synthesizing compound 18, compound 69 was prepared from 4-methylaminobenzaldehyde and 4-nitrobenzaldehyde, and column chromatography separation was carried out using dichloromethane, petroleum ether and ethyl acetate with a volume ratio of 5:15:2 as a developing solvent, to obtain 27.9 mg of red crystalline solid with a yield of 9.9%. The structure was as follows: ¹H NMR (400 MHz, CDCl₃) δ 8.69 (s, 1H), 8.60 (s, 1H), 8.28 (d, J=8.8 Hz, 2H), 7.98 (d, J=8.8 Hz, 2H), 7.71 (d, J=8.2 Hz, 2H), 6.63 (d, J=8.7 Hz, 2H), 2.92 (s, 3H).

### Example 70: Synthesis of Compound 70

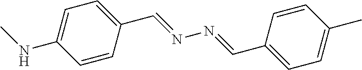

According to the method for synthesizing compound 18, compound 70 was prepared from 4-methylaminobenzaldehyde and 4-methylbenzaldehyde, and column chromatography separation was carried out using dichloromethane, petroleum ether and ethyl acetate with a volume ratio of 10:5:1 as a developing solvent, to obtain 102.4 mg of light yellow crystalline solid with a yield of 40.7%. The structure was as follows: ¹H NMR (400 MHz, CDCl₃) δ 8.63 (s, 1H), 8.58 (s, 1H), 7.70 (dd, J=12.5, 8.3 Hz, 4H), 7.25 (d, J=2.8 Hz, 2H), 6.62 (d, J=8.7 Hz, 2H), 4.15 (s, 1H), 2.89 (s, 3H), 2.40 (s, 3H).

### Example 71: Synthesis of Compound 71

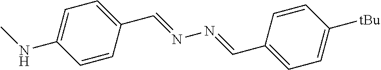 [SMILES: CNc1ccc(/C=N/N=C/c2ccc(C(C)(C)C)cc2)cc1]

According to the method for synthesizing compound 18, compound 71 was prepared from 4-methylaminobenzaldehyde and 4-tert-butylbenzaldehyde, and column chromatography separation was carried out using dichloromethane, petroleum ether and ethyl acetate with a volume ratio of 10:20:1 as a developing solvent, to obtain 25.8 mg of yellow crystalline solid with a yield of 8.8%. The structure was as follows: ¹H NMR (400 MHz, CDCl₃) δ 8.64 (s, 1H), 8.58 (s, 1H), 7.75 (d, J=8.3 Hz, 2H), 7.69 (d, J=8.3 Hz, 2H), 7.46 (d, J=8.3 Hz, 2H), 6.62 (d, J=8.7 Hz, 2H), 2.90 (s, 3H), 1.35 (s, 9H).

### Example 72: Synthesis of Compound 72

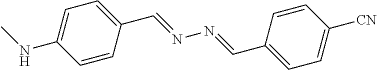 [SMILES: CNc1ccc(/C=N/N=C/c2ccc(C#N)cc2)cc1]

According to the method for synthesizing compound 18, compound 72 was prepared from 4-methylaminobenzaldehyde and 4-cyanobenzaldehyde, and column chromatography separation was carried out using petroleum ether and ethyl acetate with a volume ratio of 4:1 as a developing solvent, to obtain 62.2 mg of orange crystalline solid with a yield of 23.7%. The structure was as follows: ¹H NMR (400 MHz, CDCl₃) δ 8.64 (s, 1H), 8.58 (s, 1H), 7.91 (d, J=8.3 Hz, 2H), 7.71 (d, J=8.3 Hz, 4H), 6.63 (d, J=8.6 Hz, 2H), 4.25 (s, 1H), 2.91 (s, 3H).

### Example 73: Synthesis of Compound 73

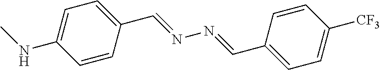 [SMILES: CNc1ccc(/C=N/N=C/c2ccc(C(F)(F)F)cc2)cc1]

According to the method for synthesizing compound 18, compound 73 was prepared from 4-methylaminobenzaldehyde and 4-trifluoromethylbenzaldehyde, and column chromatography separation was carried out using dichloromethane, petroleum ether and ethyl acetate with a volume ratio of 10:15:2 as a developing solvent, to obtain 27.8 mg of yellow crystalline solid with a yield of 9.1%. The structure was as follows: 1H NMR (400 MHz, CDCl₃) δ 8.67 (s, 1H), 8.59 (s, 1H), 7.93 (d, J=8.1 Hz, 2H), 7.69 (t, J=8.2 Hz, 4H), 6.63 (d, J=8.7 Hz, 2H), 4.20 (s, 1H), 2.91 (s, 3H).

### Example 74: Synthesis of Compound 74

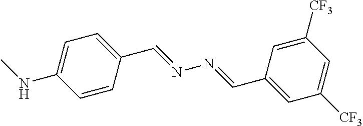 [SMILES: CNc1ccc(/C=N/N=C/c2cc(C(F)(F)F)cc(C(F)(F)F)c2)cc1]

According to the method for synthesizing compound 18, compound 74 was prepared from 4-methylaminobenzaldehyde and 3,5-bistrifluoromethylbenzaldehyde, and column chromatography separation was carried out using dichloromethane, petroleum ether and ethyl acetate with a volume ratio of 155:15:2 as a developing solvent, to obtain 28.8 mg of yellow crystalline solid with a yield of 7.7%. The structure was as follows: ¹H NMR (400 MHz, CDCl₃) δ 8.72 (s, 1H), 8.60 (s, 1H), 8.26 (s, 2H), 7.91 (s, 1H), 7.73 (s, 2H), 6.64 (d, J=8.6 Hz, 2H), 4.34-4.17 (m, 1H), 2.92 (s, 3H).

### Example 75: Synthesis of Compound 75

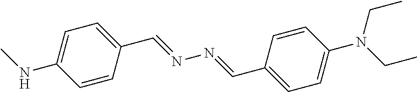 [SMILES: CCN(CC)c1ccc(/C=N/N=C/c2ccc(NC)cc2)cc1]

According to the method for synthesizing compound 18, compound 75 was prepared from 4-methylaminobenzaldehyde and 4-diethylaminobenzaldehyde, and column chromatography separation was carried out using dichloromethane, petroleum ether and ethyl acetate with a volume ratio of 10:5:1 as a developing solvent, to obtain 116.8 mg of orange crystalline solid with a yield of 37.9%. The structure was as follows: ¹H NMR (400 MHz, CDCl₃) δ 8.56 (s, 1H), 8.55 (s, 1H), 7.66 (d, J=8.5 Hz, 4H), 6.67 (d, J=8.9 Hz, 2H), 6.61 (d, J=8.7 Hz, 2H), 4.08 (s, 1H), 3.41 (q, J=7.1 Hz, 4H), 2.88 (s, 3H), 1.20 (t, J=7.1 Hz, 6H).

### Example 76: Synthesis of Compound 76

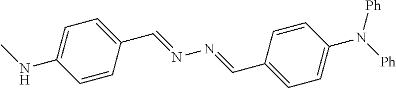 [SMILES: CNc1ccc(/C=N/N=C/c2ccc(N(c3ccccc3)c3ccccc3)cc2)cc1]

According to the method for synthesizing compound 18, compound 76 was prepared from 4-methylaminobenzaldehyde and 4-diphenylaminobenzaldehyde, and column chromatography separation was carried out using dichloromethane, petroleum ether and ethyl acetate with a volume ratio of 10:5:2 as a developing solvent, to obtain 150.9 mg of yellow crystalline solid with a yield of 37.3%. The structure was as follows: ¹H NMR (400 MHz, CDCl₃) δ 8.59 (s, 1H), 8.55 (s, 1H), 7.67 (t, J=9.1 Hz, 4H), 7.32-7.26 (m, 4H), 7.14 (d, J=7.6 Hz, 4H), 7.07 (dd, J=16.3, 8.0 Hz, 4H), 6.62 (d, J=8.7 Hz, 2H), 4.13 (s, 1H), 2.90 (s, 3H).

### Example 77: Synthesis of Compound 77

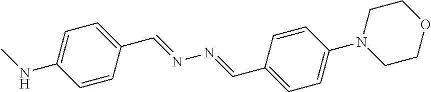 [SMILES: CNc1ccc(/C=N/N=C/c2ccc(N3CCOCC3)cc2)cc1]

According to the method for synthesizing compound 18, compound 77 was prepared from 4-methylaminobenzaldehyde and 4-(4-morpholinyl)benzaldehyde, and column chromatography separation was carried out using dichloromethane, petroleum ether and ethyl acetate with a volume ratio of 2:2:1 as a developing solvent, to obtain 98.3 mg of yellow crystalline solid with a yield of 30.5%. The structure was as follows: ¹H NMR (400 MHz, CDCl₃) δ 8.59-8.56 (m, 2H), 7.73 (d, J=8.7 Hz, 2H), 7.68 (d, J=7.5 Hz, 2H), 6.92 (d, J=8.8 Hz, 2H), 6.62 (d, J=8.7 Hz, 2H), 4.13 (s, 1H), 3.98-3.76 (m, 4H), 3.38-3.17 (m, 4H), 2.90 (s, 3H).

### Example 78: Synthesis of Compound 78

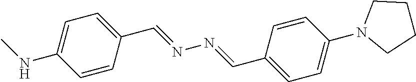 [SMILES: CNc1ccc(/C=N/N=C/c2ccc(N3CCCC3)cc2)cc1]

According to the method for synthesizing compound 18, compound 78 was prepared from 4-methylaminobenzaldehyde and 4-(1-pyrrolidinyl)benzaldehyde, and column chromatography separation was carried out using dichloromethane, petroleum ether and ethyl acetate with a volume ratio of 15:5:1 as a developing solvent, to obtain 24.9 mg of orange crystalline solid with a yield of 8.1%. The structure was as follows: ¹H NMR (400 MHz, CDCl₃) δ 8.55 (s, 2H), 7.65 (d, J=8.6 Hz, 4H), 6.58 (dd, J=15.8, 8.8 Hz, 4H), 3.35 (t, J=6.5 Hz, 4H), 2.88 (s, 3H), 2.04-1.99 (m, 4H).

### Example 79: Synthesis of Compound 79

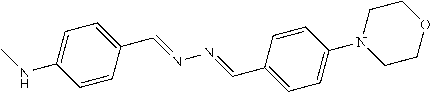 [SMILES: CNc1ccc(/C=N/N=C/c2ccc(N3CCOCC3)cc2)cc1]

According to the method for synthesizing compound 18, compound 79 was prepared from 4-methylaminobenzaldehyde and 4-(1-piperidinyl)benzaldehyde, and column chromatography separation was carried out using dichloromethane, petroleum ether and ethyl acetate with a volume ratio of 10:15:2 as a developing solvent, to obtain 123.6 mg of yellow crystalline solid with a yield of 38.6%. The structure was as follows: ¹H NMR (400 MHz, CDCl₃) δ 8.55 (s, 2H), 7.67 (t, J=9.3 Hz, 4H), 6.91 (d, J=8.9 Hz, 2H), 6.60 (d, J=8.8 Hz, 2H), 3.32-3.26 (m, 4H), 2.88 (s, 3H), 1.72-1.66 (m, 4H), 1.62 (dd, J=10.3, 5.4 Hz, 2H).

### Example 80: Synthesis of Compound 80

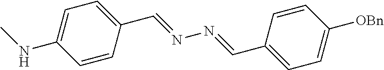 [SMILES: CNc1ccc(/C=N/N=C/c2ccc(OCc3ccccc3)cc2)cc1]

According to the method for synthesizing compound 18, compound 80 was prepared from 4-methylaminobenzaldehyde and 4-benzyloxybenzaldehyde, and column chromatography separation was carried out using dichloromethane, petroleum ether and ethyl acetate with a volume ratio of 10:10:1 as a developing solvent, to obtain 111.2 mg of yellow crystalline solid with a yield of 32.4%. The structure was as follows: ¹H NMR (400 MHz, CDCl₃) δ 8.61 (d, J=5.7 Hz, 1H), 8.56 (s, 1H), 7.77 (dd, J=7.6, 4.1 Hz, 2H), 7.69 (d, J=7.8 Hz, 2H), 7.40 (dt, J=8.7, 6.9 Hz, 5H), 7.06-7.01 (m, 2H), 6.62 (d, J=8.7 Hz, 2H), 5.12 (s, 2H), 4.13 (s, 1H), 2.90 (s, 3H).

### Example 81: Synthesis of Compound 81

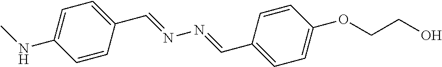 [SMILES: CNc1ccc(/C=N/N=C/c2ccc(OCCO)cc2)cc1]

According to the method for synthesizing compound 18, compound 81 was prepared from 4-methylaminobenzaldehyde and 4-(2-hydroxyethoxy)benzaldehyde, and column chromatography separation was carried out using dichloromethane, petroleum ether and ethyl acetate with a volume ratio of 10:5:3 as a developing solvent, to obtain 84.4 mg of yellow crystalline solid with a yield of 28.4%. The structure was as follows: ¹H NMR (400 MHz, CDCl₃) δ 8.59 (s, 1H), 8.55 (s, 1H), 7.75 (d, J=8.8 Hz, 2H), 7.67 (d, J=8.5 Hz, 2H), 6.95 (d, J=8.7 Hz, 2H), 6.61 (d, J=8.7 Hz, 2H), 4.14-4.10 (m, 2H), 3.99-3.95 (m, 2H), 2.88 (s, 3H).

### Example 82: Synthesis of Compound 82

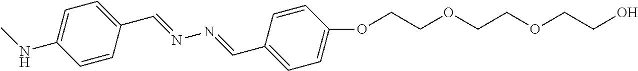 [SMILES: CNc1ccc(/C=N/N=C/c2ccc(OCCOCCOCCO)cc2)cc1]

According to the method for synthesizing compound 18, compound 82 was prepared from 4-methylaminobenzaldehyde and 4-{2-[2-(2-hydroxyethoxy)ethoxy]ethoxy}benzaldehyde, and column chromatography separation was carried out using dichloromethane and ethyl acetate with a volume ratio of 3:1 as a developing solvent, to obtain 30.8 mg of yellow waxy solid with a yield of 8.0%. The structure was as follows: ¹H NMR (400 MHz, CDCl₃) δ 8.61 (s, 1H), 8.54 (s, 1H), 7.74 (d, J=8.7 Hz, 2H), 7.69 (dd, J=9.3, 5.9 Hz, 2H), 6.96 (d, J=8.7 Hz, 2H), 6.61 (d, J=8.7 Hz, 2H), 4.19-4.16 (m, 2H), 3.89-3.86 (m, 2H), 3.75-3.68 (m, 6H), 3.63-0.59 (m, 2H), 2.89 (s, 3H).

### Example 83: Synthesis of Compound 83

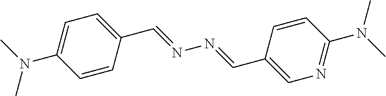

According to the method for synthesizing compound 18, compound 83 was prepared from 4-dimethylaminobenzaldehyde and 6-dimethylaminonicotinaldehyde, and column chromatography separation was carried out using petroleum ether and ethyl acetate with a volume ratio of 4:1 as a developing solvent, to obtain 100.0 mg of yellow crystalline solid with a yield of 33.8%. The structure was as follows: ¹H NMR (400 MHz, CDCl₃) δ 8.55 (s, 1H), 8.54 (s, 1H), 8.37 (d, J=2.1 Hz, 1H), 8.07 (d, J=5.4 Hz, 1H), 7.70 (d, J=6.2 Hz, 2H), 6.71 (d, J=9.0 Hz, 2H), 6.57 (d, J=8.9 Hz, 1H), 3.17 (s, 6H), 3.03 (s, 6H).

### Example 84: Synthesis of Compound 84

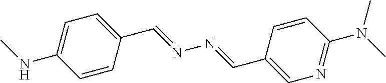

According to the method for synthesizing compound 18, compound 84 was prepared from 4-methylaminobenzaldehyde and 6-dimethylaminonicotinaldehyde, and column chromatography separation was carried out using petroleum ether and ethyl acetate with a volume ratio of 3:1 as a developing solvent, to obtain 50.5 mg of yellow crystalline solid with a yield of 17.9%. The structure was as follows: ¹H NMR (400 MHz, CDCl₃) δ 8.55 (s, 1H), 8.53 (s, 1H), 8.37 (d, J=2.2 Hz, 1H), 8.06 (d, J=7.8 Hz, 1H), 7.66 (d, J=8.4 Hz, 2H), 6.61 (d, J=8.7 Hz, 2H), 6.57 (d, J=9.0 Hz, 1H), 4.10 (s, 1H), 3.16 (s, 6H), 2.88 (s, 3H).

### Example 85: Synthesis of Compound 85

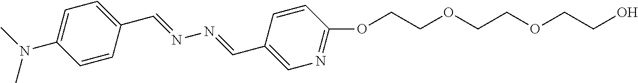 [SMILES: CN(C)c1ccc(/C=N/N=C/c2ccc(OCCOCCOCCO)nc2)cc1]

According to the method for synthesizing compound 18, compound 85 was prepared from 4-dimethylaminobenzaldehyde and 6-{2-[2-(2-hydroxyethoxy)ethoxy]ethoxy}nicotinaldehyde, and column chromatography separation was carried out using dichloromethane and ethyl acetate with a volume ratio of 1:3 as a developing solvent, to obtain 50.4 mg of brown waxy solid with a yield of 12.6%. The structure was as follows: ¹H NMR (400 MHz, CDCl₃) δ 8.60 (s, 1H), 8.55 (s, 1H), 8.37 (s, 1H), 8.16 (dd, J=8.7, 2.3 Hz, 1H), 7.70 (dd, J=5.6, 3.3 Hz, 2H), 6.84 (d, J=8.7 Hz, 1H), 6.71 (d, J=8.1 Hz, 2H), 4.55-4.51 (m, 2H), 3.90-3.83 (m, 2H), 3.72 (dd, J=8.6, 3.7 Hz, 6H), 3.63-3.59 (m, 2H), 3.04 (s, 6H).

### Example 86: Synthesis of Compound 86

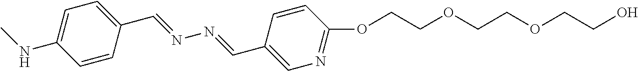 [SMILES: CNc1ccc(/C=N/N=C/c2ccc(OCCOCCOCCO)nc2)cc1]

According to the method for synthesizing compound 18, compound 86 was prepared from 4-methylaminobenzaldehyde and 6-{2-[2-(2-hydroxyethoxy)ethoxy]ethoxy}nicotinaldehyde, and column chromatography separation was carried out using dichloromethane and ethyl acetate with a volume ratio of 1:4 as a developing solvent, to obtain 40.3 mg of brown waxy solid with a yield of 10.4%. The structure was as follows: ¹H NMR (400 MHz, CDCl₃) δ 8.59 (s, 1H), 8.53 (s, 1H), 8.37 (d, J=2.2 Hz, 1H), 8.16 (dd, J=8.7, 2.3 Hz, 1H), 7.66 (d, J=8.4 Hz, 2H), 6.84 (d, J=8.7 Hz, 1H), 6.61 (d, J=8.8 Hz, 2H), 4.53 (dd, J=5.4, 4.1 Hz, 2H), 3.88-3.84 (m, 2H), 3.74-3.67 (m, 6H), 3.61 (dd, J=5.2, 3.8 Hz, 2H), 2.89 (s, 3H).

### Example 87: Synthesis of Compound 87

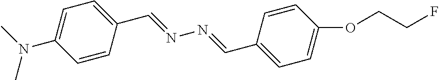 [SMILES: CN(C)c1ccc(/C=N/N=C/c2ccc(OCCF)cc2)cc1]

According to the method for synthesizing compound 18, compound 87 was prepared from 4-dimethylaminobenzaldehyde and 4-(2-fluoroethoxy)benzaldehyde, and column chromatography separation was carried out using dichloromethane, petroleum ether and ethyl acetate with a volume ratio of 10:5:1 as a developing solvent, to obtain 84.0 mg of yellow crystalline solid with a yield of 26.8%. The structure was as follows: ¹H NMR (400 MHz, CDCl₃) δ 8.62 (s, 1H), 8.58 (s, 1H), 7.78 (d, J=8.7 Hz, 2H), 7.71 (d, J=8.2 Hz, 2H), 6.98 (d, J=8.7 Hz, 2H), 6.72 (d, J=9.0 Hz, 2H), 4.87-4.81 (m, 1H), 4.75-4.70 (m, 1H), 4.34-4.27 (m, 1H), 4.26-4.20 (m, 1H), 3.05 (s, 6H).

### Example 88: Synthesis of Compound 88

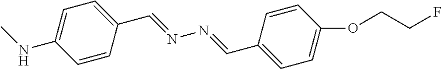 [SMILES: CNc1ccc(/C=N/N=C/c2ccc(OCCF)cc2)cc1]

According to the method for synthesizing compound 18, compound 88 was prepared from 4-methylaminobenzaldehyde and 4-(2-fluoroethoxy)benzaldehyde, and column chromatography separation was carried out using dichloromethane, petroleum ether and ethyl acetate with a volume ratio of 10:5:2 as a developing solvent, to obtain 29.9 mg of yellow crystalline solid with a yield of 10.0%. The structure was as follows: ¹H NMR (400 MHz, CDCl₃) δ 8.61 (s, 1H), 8.56 (s, 1H), 7.78 (dd, J=8.6, 6.3 Hz, 2H), 7.69 (d, J=7.9 Hz, 2H), 7.01-6.95 (m, 2H), 6.62 (d, J=8.8 Hz, 2H), 4.86-4.82 (m, 1H), 4.77-4.67 (m, 1H), 4.35-4.28 (m, 1H), 4.27-4.19 (m, 1H). 2.90 (s, 3H).

### Example 89: Synthesis of Compound 89

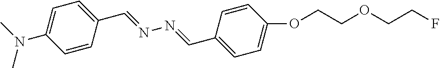 [SMILES: CN(C)c1ccc(/C=N/N=C/c2ccc(OCCOCCF)cc2)cc1]

According to the method for synthesizing compound 18, compound 89 was prepared from 4-dimethylaminobenzaldehyde and 4-[2-(2-fluoroethoxy)ethoxy]benzaldehyde, and column chromatography separation was carried out using dichloromethane, petroleum ether and ethyl acetate with a volume ratio of 15:5:1 as a developing solvent, to obtain 81.8 mg of light yellow crystalline solid with a yield of 22.9%. The structure was as follows: ¹H NMR (400 MHz, CDCl₃) δ 8.61 (d, J=2.3 Hz, 1H), 8.57 (s, 1H), 7.76 (d, J=8.8 Hz, 2H), 7.71 (d, J=8.3 Hz, 2H), 6.97 (d, J=8.8 Hz, 2H), 6.72 (d, J=9.0 Hz, 2H), 4.68-4.64 (m, 1H), 4.59-4.51 (m, 1H), 4.24-4.16 (m, 2H), 3.91 (dd, J=8.4, 3.8 Hz, 2H), 3.89-3.84 (m, 1H), 3.82-3.76 (m, 1H), 3.04 (s, 6H).

### Example 90: Synthesis of Compound 90

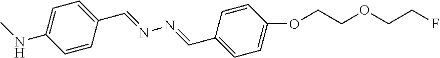 [SMILES: CNc1ccc(/C=N/N=C/c2ccc(OCCOCCF)cc2)cc1]

According to the method for synthesizing compound 18, compound 90 was prepared from 4-methylaminobenzaldehyde and 4-[2-(2-fluoroethoxy)ethoxy]benzaldehyde, and column chromatography separation was carried out using petroleum ether and ethyl acetate with a volume ratio of 4:1 as a developing solvent, to obtain 38.3 mg of yellow crystalline solid with a yield of 11.2%. The structure was as follows: ¹H NMR (400 MHz, CDCl₃) δ 8.64-8.54 (m, 2H), 7.73 (dd, J=24.1, 7.0 Hz, 4H), 6.97 (d, J=8.7 Hz, 2H), 6.62 (d, J=8.6 Hz, 2H), 4.69-4.62 (m, 1H), 4.60-4.50 (m, 1H), 4.25-4.15 (m, 2H), 3.95-3.84 (m, 3H), 3.82-3.74 (m, 1H), 2.90 (s, 3H).

### Example 91: Synthesis of Compound 91

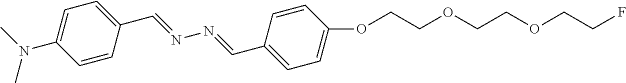 [SMILES: CN(C)c1ccc(/C=N/N=C/c2ccc(OCCOCCOCCF)cc2)cc1]

According to the method for synthesizing compound 18, compound 91 was prepared from 4-dimethylaminobenzaldehyde and 4-{2-[2-(2-fluoroethoxy)ethoxy]ethoxy}benzaldehyde, and column chromatography separation was carried out using dichloromethane, petroleum ether and ethyl acetate with a volume ratio of 5:3:2 as a developing solvent, to obtain 156.0 mg of light yellow crystalline solid with a yield of 38.8%. The structure was as follows: ¹H NMR (400 MHz, CDCl₃) δ 8.61 (s, 1H), 8.56 (s, 1H), 7.75 (d, J=8.7 Hz, 4H), 6.96 (d, J=8.7 Hz, 2H), 6.72 (d, J=8.8 Hz, 2H), 4.65-4.60 (m, 1H), 4.53-4.48 (m, 1H), 4.22-4.17 (m, 2H), 3.91-3.86 (m, 2H), 3.80-3.71 (m, 6H), 3.05 (s, 6H).

### Example 92: Synthesis of Compound 92

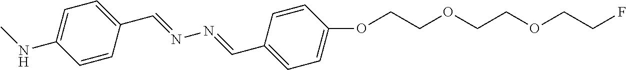 [SMILES: CNc1ccc(/C=N/N=C/c2ccc(OCCOCCOCCF)cc2)cc1]

According to the method for synthesizing compound 18, compound 92 was prepared from 4-methylaminobenzaldehyde and 4-{2-[2-(2-fluoroethoxy)ethoxy]ethoxy}benzaldehyde, and column chromatography separation was carried out using dichloromethane, petroleum ether and ethyl acetate with a volume ratio of 5:3:3 as a developing solvent, to obtain 108.3 mg of yellow crystalline solid with a yield of 27.9%. The structure was as follows: ¹H NMR (400 MHz, CDCl₃) δ 8.60 (s, 1H), 8.56 (s, 1H), 7.75 (d, J=8.7 Hz, 2H), 7.67 (d, J=8.2 Hz, 2H), 6.96 (d, J=8.7 Hz, 2H), 6.62 (d, J=8.7 Hz, 2H), 4.67-4.60 (m, 1H), 4.54-4.47 (m, 1H), 4.24-4.17 (m, 2H), 3.93-3.86 (m, 2H), 3.82-3.69 (m, 6H), 2.89 (s, 3H).

### Example 93: Synthesis of Compound 93

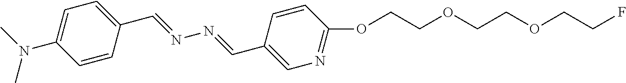 [SMILES: CN(C)c1ccc(/C=N/N=C/c2ccc(OCCOCCOCCF)nc2)cc1]

According to the method for synthesizing compound 18, compound 93 was prepared from 4-dimethylaminobenzaldehyde and 6-{2-[2-(2-fluoroethoxy)ethoxy]ethoxy}nicotinaldehyde, and column chromatography separation was carried out using dichloromethane, petroleum ether and ethyl acetate with a volume ratio of 1:1:1 as a developing solvent, to obtain 62.5 mg of light yellow crystalline solid with a yield of 15.5%. The structure was as follows: ¹H NMR (400 MHz, CDCl₃) δ 8.61 (s, 1H), 8.56 (s, 1H), 8.38 (d, J=2.1 Hz, 1H), 8.17 (dd, J=8.7, 2.3 Hz, 1H), 7.71 (d, J=7.4 Hz, 2H), 6.84 (d, J=8.7 Hz, 1H), 6.72 (d, J=8.9 Hz, 2H), 4.65-4.60 (m, 1H), 4.57-4.52 (m, 2H), 4.52-4.48 (m, 1H), 3.91-3.85 (m, 2H), 3.81-3.77 (m, 1H), 3.76-3.69 (m, 5H), 3.05 (s, 6H).

### Example 94: Synthesis of Compound 94

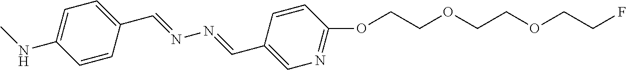 [SMILES: CNc1ccc(/C=N/N=C/c2ccc(OCCOCCOCCF)nc2)cc1]

According to the method for synthesizing compound 18, compound 94 was prepared from 4-methylaminobenzaldehyde and 6-{2-[2-(2-fluoroethoxy)ethoxy]ethoxy}nicotinaldehyde, and column chromatography separation was carried out using dichloromethane, petroleum ether and ethyl acetate with a volume ratio of 1:1:1 as a developing solvent, to obtain 63.0 mg of yellow crystalline solid with a yield of 16.2%. The structure was as follows: ¹H NMR (400 MHz, CDCl₃) δ 8.62 (s, 1H), 8.52 (s, 1H), 8.37 (d, J=2.2 Hz, 1H), 8.16 (dd, J=8.7, 2.3 Hz, 1H), 7.68 (s, 2H), 6.83 (d, J=8.7 Hz, 1H), 6.61 (d, J=8.8 Hz, 2H), 4.63-4.59 (m, 1H), 4.55-4.52 (m, 2H), 4.49 (dd, J=5.8, 2.6 Hz, 1H), 3.89-3.84 (m, 2H), 3.80-3.76 (m, 1H), 3.75-3.68 (m, 5H), 2.90 (s, 3H).

### Example 95: Synthesis of Compound 95

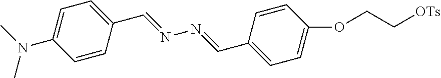 [SMILES: Cc1ccc(S(=O)(=O)OCCOc2ccc(/C=N/N=C/c3ccc(N(C)C)cc3)cc2)cc1]

According to the method for synthesizing compound 18, compound 95 was prepared from 4-dimethylaminobenzaldehyde and 4-{2-[(4-methylphenyl)sulfonyl]ethoxy}benzaldehyde, and column chromatography separation was carried out using petroleum ether and ethyl acetate with a volume ratio of 3:1 as a developing solvent, to obtain 52.3 mg of light yellow crystalline solid with a yield of 11.2%. The structure was as follows: ¹H NMR (400 MHz, CDCl₃) δ 8.60 (s, 1H), 8.57 (s, 1H), 7.82 (d, J=8.3 Hz, 2H), 7.72 (d, J=8.7 Hz, 4H), 7.34 (d, J=8.1 Hz, 2H), 6.81 (d, J=8.8 Hz, 2H), 6.72 (d, J=9.0 Hz, 2H), 4.39 (dd, J=5.5, 3.9 Hz, 2H), 4.19 (dd, J=5.5, 3.9 Hz, 2H), 3.05 (s, 6H), 2.44 (s, 3H).

### Example 96: Synthesis of Compound 96

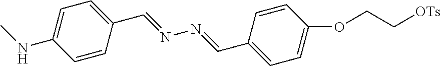 [SMILES: CNc1ccc(/C=N/N=C/c2ccc(OCCOS(=O)(=O)c3ccc(C)cc3)cc2)cc1]

According to the method for synthesizing compound 18, compound 96 was prepared from 4-methylaminobenzaldehyde and 4-{2-[(4-methylphenyl)sulfonyl]ethoxy}benzaldehyde, and column chromatography separation was carried out using dichloromethane, petroleum ether and ethyl acetate with a volume ratio of 30:103 as a developing solvent, to obtain 152.0 mg of light yellow crystalline solid with a yield of 33.7%. The structure was as follows: ¹H NMR (600 MHz, CDCl₃) δ 8.63 (s, 1H), 8.51 (s, 1H), 7.80 (d, J=8.1 Hz, 2H), 7.71 (d, J=8.5 Hz, 4H), 7.32 (d, J=8.0 Hz, 2H), 6.80 (d, J=8.5 Hz, 2H), 6.62 (d, J=8.3 Hz, 2H), 4.43-4.32 (m, 2H), 4.24-4.14 (m, 2H), 2.90 (s, 3H), 2.44 (s, 3H).

### Example 97: Synthesis of Compound 97

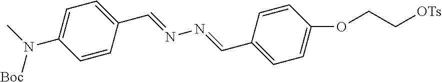 [SMILES: Cc1ccc(S(=O)(=O)OCCOc2ccc(/C=N/N=C/c3ccc(N(C)C(=O)OC(C)(C)C)cc3)cc2)cc1]

Compound 96 (55.8 mg, 0.12 mmol) was dissolved in 12 mL of anhydrous tetrahydrofuran, and BOC anhydride (261.8 mg, 1.2 mmol) was added slowly into the reaction flask, and the reaction was carried out for 12 hours at 90° C. under reflux. TLC was used for monitoring until the reaction was carried out completely. The solvent was removed by rotary evaporation, and column chromatography separation was carried out using petroleum ether and ethyl acetate with a volume ratio of 3:1 as a developing solvent, to obtain 65.0 mg of yellow solid with a yield of 95.4%. The structure was as follows: ¹H NMR (400 MHz, CDCl₃) δ 8.62 (d, J=9.5 Hz, 2H), 7.87-7.68 (m, 6H), 7.34 (dd, J=8.4, 2.0 Hz, 4H), 6.84 (d, J=8.8 Hz, 2H), 4.40 (dd, J=5.5, 3.8 Hz, 2H), 4.25-4.17 (m, 2H), 3.30 (s, 3H), 2.45 (s, 3H), 1.47 (s, 9H).

### Example 98: Synthesis of Compound 98

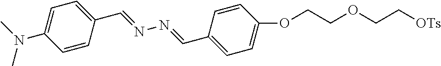 [SMILES: Cc1ccc(S(=O)(=O)OCCOCCOc2ccc(/C=N/N=C/c3ccc(N(C)C)cc3)cc2)cc1]

According to the method for synthesizing compound 18, compound 98 was prepared from 4-dimethylaminobenzaldehyde and 4-[2-{2-[(4-methylphenyl)sulfonyl]ethoxy}ethoxy]benzaldehyde, and column chromatography separation was carried out using dichloromethane, petroleum ether and ethyl acetate with a volume ratio of 10:5:1 as a developing solvent, to obtain 156.1 mg of light yellow crystalline solid with a yield of 30.6%. The structure was as follows: ¹H NMR (400 MHz, CDCl₃) δ 8.61 (s, 1H), 8.57 (s, 1H), 7.83-7.67 (m, 6H), 7.30 (d, J=8.1 Hz, 2H), 6.93 (d, J=8.7 Hz, 2H), 6.72 (d, J=8.8 Hz, 2H), 4.22-4.18 (m, 2H), 4.11-4.06 (m, 2H), 3.83-3.79 (m, 2H), 3.78-3.74 (m, 2H), 3.05 (s, 6H), 2.41 (s, 3H).

### Example 99: Synthesis of Compound 99

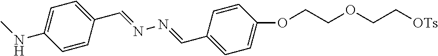 [SMILES: CNc1ccc(/C=N/N=C/c2ccc(OCCOCCOS(=O)(=O)c3ccc(C)cc3)cc2)cc1]

According to the method for synthesizing compound 18, compound 99 was prepared from 4-methylaminobenzaldehyde and 4-[2-{2-[(4-methylphenyl)sulfonyl]ethoxy}ethoxy]benzaldehyde, and column chromatography separation was carried out using dichloromethane, petroleum ether and ethyl acetate with a volume ratio of 1:2:1 as a developing solvent, to obtain 127.5 mg of yellow waxy solid with a yield of 25.7%. The structure was as follows: ¹H NMR (400 MHz, CDCl₃) δ 8.61 (s, 1H), 8.56 (s, 1H), 7.77 (dd, J=15.8, 8.5 Hz, 4H), 7.68 (d, J=7.6 Hz, 2H), 7.30 (d, J=8.1 Hz, 2H), 6.93 (d, J=8.7 Hz, 2H), 6.62 (d, J=8.7 Hz, 2H), 4.23-4.18 (m, 2H), 4.12-4.06 (m, 2H), 3.85-3.75 (m, 4H), 2.90 (s, 3H), 2.40 (s, 3H).

### Example 100: Synthesis of Compound 100

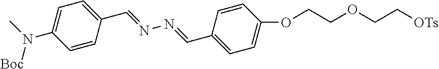 [SMILES: Cc1ccc(S(=O)(=O)OCCOCCOc2ccc(/C=N/N=C/c3ccc(N(C)C(=O)OC(C)(C)C)cc3)cc2)cc1]

According to the method for synthesizing compound 97, compound 100 was prepared from compound 99, and column chromatography separation was carried out using petroleum ether and ethyl acetate with a volume ratio of 2:1 as a developing solvent, to obtain 120.0 mg of light yellow solid with a yield of 96.5%. The structure was as follows: ¹H NMR (400 MHz, CDCl₃) δ 8.65 (s, 1H), 8.62 (s, 1H), 7.79 (dd, J=8.5, 3.7 Hz, 6H), 7.34-7.28 (m, 4H), 6.94 (d, J=8.8 Hz, 2H), 4.19 (dd, J=5.4, 4.2 Hz, 2H), 4.12-4.07 (m, 2H), 3.81 (dd, J=5.4, 4.0 Hz, 2H), 3.76 (dd, J=5.4, 4.1 Hz, 2H), 3.29 (s, 3H), 2.40 (s, 3H), 1.47 (s, 9H).

### Example 101: Synthesis of Compound 101

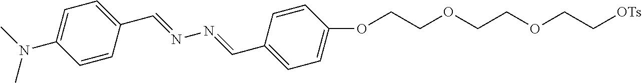 [SMILES: Cc1ccc(S(=O)(=O)OCCOCCOCCOc2ccc(/C=N/N=C/c3ccc(N(C)C)cc3)cc2)cc1]

According to the method for synthesizing compound 18, compound 101 was prepared from 4-dimethylaminobenzaldehyde and 4-{2-[2-{2-[(4-methylphenyl)sulfonyl]ethoxy}ethoxy]ethoxy} benzaldehyde, and column chromatography separation was carried out using dichloromethane, petroleum ether and ethyl acetate with a volume ratio of 2:3:1 as a developing solvent, to obtain 186.3 mg of light yellow crystalline solid with a yield of 33.6%. The structure was as follows: ¹H NMR (400 MHz, CDCl₃) δ 8.62 (s, 1H), 8.56 (s, 1H), 7.77 (dd, J=17.4, 8.5 Hz, 6H), 7.32 (d, J=8.1 Hz, 2H), 6.95 (d, J=8.8 Hz, 2H), 6.72 (d, J=9.0 Hz, 2H), 4.16 (dd, J=5.2, 4.4 Hz, 4H), 3.91-3.80 (m, 2H), 3.73-3.65 (m, 4H), 3.62 (dd, J=5.8, 2.8 Hz, 2H), 3.05 (s, 6H), 2.42 (s, 3H).

### Example 102: Synthesis of Compound 102

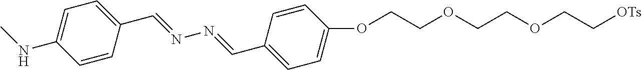 [SMILES: CNc1ccc(/C=N/N=C/c2ccc(OCCOCCOCCOS(=O)(=O)c3ccc(C)cc3)cc2)cc1]

According to the method for synthesizing compound 18, compound 102 was prepared from 4-methylaminobenzaldehyde and 4-{2-[2-{2-[(4-methylphenyl)sulfonyl]ethoxy}ethoxy]ethoxy} benzaldehyde, and column chromatography separation was carried out using dichloromethane, petroleum ether and ethyl acetate with a volume ratio of 8:5:3 as a developing solvent, to obtain 156.0 mg of yellow waxy solid with a yield of 28.9%. The structure was as follows: ¹H NMR (400 MHz, CDCl₃) δ 8.61-8.54 (m, 2H), 7.85-7.57 (m, 6H), 7.32 (d, J=8.1 Hz, 2H), 6.95 (d, J=8.7 Hz, 2H), 6.62 (d, J=8.4 Hz, 2H), 4.29-4.00 (m, 4H), 3.88-3.79 (m, 2H), 3.75-3.53 (m, 6H), 2.90 (s, 3H), 2.42 (s, 3H).

### Example 103: Synthesis of Compound 103

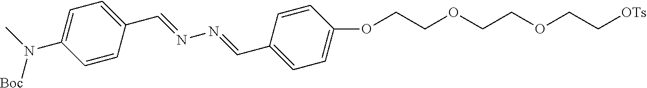 [SMILES: Cc1ccc(S(=O)(=O)OCCOCCOCCOc2ccc(/C=N/N=C/c3ccc(N(C)C(=O)OC(C)(C)C)cc3)cc2)cc1]

According to the method for synthesizing compound 97, compound 103 was prepared from compound 102, and column chromatography separation was carried out using petroleum ether and ethyl acetate with a volume ratio of 2:1 as a developing solvent, to obtain 197.3 mg of light yellow solid with a yield of 98.4%. The structure was as follows: ¹H NMR (400 MHz, CDCl₃) δ 8.63 (d, J=6.9 Hz, 2H), 7.92-7.63 (m, 6H), 7.33 (dd, J=8.2, 3.2 Hz, 4H), 6.97 (d, J=8.7 Hz, 2H), 4.16 (dd, J=9.0, 4.1 Hz, 4H), 4.01-3.79 (m, 2H), 3.79-3.59 (m, 6H), 3.30 (s, 3H), 2.43 (s, 3H), 1.47 (s, 9H).

### Example 104: Synthesis of Compound 104

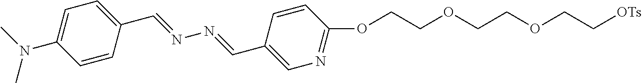 [SMILES: Cc1ccc(S(=O)(=O)OCCOCCOCCOc2ccc(/C=N/N=C/c3ccc(N(C)C)cc3)cn2)cc1]

According to the method for synthesizing compound 18, compound 104 was prepared from 4-dimethylaminobenzaldehyde and 6-{2-[2-{2-[(4-methylphenyl)sulfonyl]ethoxy}ethoxy]ethoxy} nicotinaldehyde, and column chromatography separation was carried out using dichloromethane, petroleum ether and ethyl acetate with a volume ratio of 2:2:1 as a developing solvent, to obtain 115.0 mg of yellow crystalline solid with a yield of 20.7%. The structure was as follows: ¹H NMR (400 MHz, CDCl₃) δ 8.61 (s, 1H), 8.55 (s, 1H), 8.38 (d, J=1.9 Hz, 1H), 8.16 (dd, J=8.7, 2.2 Hz, 1H), 7.80 (d, J=8.3 Hz, 2H), 7.70 (s, 2H), 7.33 (d, J=8.1 Hz, 2H), 6.83 (d, J=8.7 Hz, 1H), 6.72 (d, J=8.8 Hz, 2H), 4.56-4.48 (m, 2H), 4.20-4.13 (m, 2H), 3.86-3.80 (m, 2H), 3.72-3.67 (m, 2H), 3.63 (ddd, J=5.3, 4.4, 2.0 Hz, 4H), 3.05 (s, 6H), 2.43 (s, 3H).

### Example 105: Synthesis of Compound 105

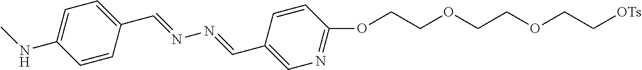 [SMILES: CNc1ccc(/C=N/N=C/c2ccc(OCCOCCOCCOS(=O)(=O)c3ccc(C)cc3)nc2)cc1]

According to the method for synthesizing compound 18, compound 105 was prepared from 4-methylaminobenzaldehyde and 6-{2-[2-{2-[(4-methylphenyl)sulfonyl]ethoxy}ethoxy]ethoxy} nicotinaldehyde, and column chromatography separation was carried out using dichloromethane, petroleum ether and ethyl acetate with a volume ratio of 5:5:4 as a developing solvent, to obtain 123.0 mg of yellow waxy solid with a yield of 22.1%. The structure was as follows: ¹H NMR (400 MHz, CDCl₃) δ 8.60 (s, 1H), 8.54 (s, 1H), 8.37 (d, J=2.2 Hz, 1H), 8.16 (dd, J=8.7, 2.3 Hz, 1H), 7.79 (d, J=8.3 Hz, 2H), 7.67 (d, J=8.5 Hz, 2H), 7.32 (d, J=8.1 Hz, 2H), 6.83 (d, J=8.7 Hz, 1H), 6.61 (d, J=8.7 Hz, 2H), 4.55-4.47 (m, 2H), 4.20-4.13 (m, 2H), 3.86-79 (m, 2H), 3.73-3.67 (m, 2H), 3.62 (ddt, J=5.8, 3.9, 2.8 Hz, 4H), 2.89 (s, 3H), 2.43 (s, 3H).

### Example 106: Synthesis of Compound 106

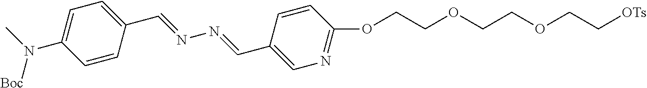 [SMILES: Cc1ccc(S(=O)(=O)OCCOCCOCCOc2ccc(/C=N/N=C/c3ccc(N(C)C(=O)OC(C)(C)C)cc3)cn2)cc1]

According to the method for synthesizing compound 97, compound 106 was prepared from compound 105, and column chromatography separation was carried out using petroleum ether and ethyl acetate with a volume ratio of 2:1 as a developing solvent, to obtain 58.7 mg of light yellow solid with a yield of 89.4%. The structure was as follows: ¹H NMR (400 MHz, CDCl₃) δ 8.63 (s, 1H), 8.61 (s, 1H), 8.41 (d, J=2.2 Hz, 1H), 8.19 (dd, J=8.7, 2.3 Hz, 1H), 7.81-7.77 (m, 4H), 7.33 (t, J=8.2 Hz, 4H), 6.84 (d, J=8.7 Hz, 1H), 4.54-4.49 (m, 2H), 4.15 (dd, J=5.4, 4.3 Hz, 2H), 3.84-3.80 (m, 2H), 3.69 (dd, J=5.4, 4.3 Hz, 2H), 3.66-3.59 (m, 4H), 3.30 (s, 3H), 2.42 (s, 3H), 1.47 (s, 9H).

### Example 107: Synthesis of Compound 107

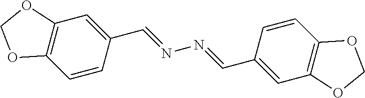 [SMILES: C(=N\N=C\c1ccc2c(c1)OCO2)/c1ccc2c(c1)OCO2]

According to the method for synthesizing compound 1, compound 107 was prepared from piperonal, and 32.1 mg of white crystalline solid was obtained with a yield of 10.8%. The structure was as follows: ¹H NMR (400 MHz, CDCl₃) δ 8.55 (s, 2H), 7.46 (d, J=1.3 Hz, 2H), 7.33-7.06 (m, 2H), 6.86 (d, J=8.0 Hz, 2H), 6.03 (s, 4H).

### Example 108: Synthesis of Compound 108

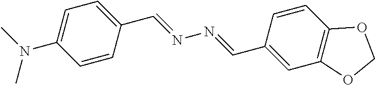 [SMILES: CN(C)c1ccc(/C=N/N=C/c2ccc3c(c2)OCO3)cc1]

According to the method for synthesizing compound 18, compound 108 was prepared from 4-dimethylaminobenzaldehyde and piperonal, and column chromatography separation was carried out using petroleum ether and ethyl acetate with a volume ratio of 11:1 as a developing solvent, to obtain 36.3 mg of yellow crystalline solid with a yield of 12.3%. The structure was as follows: ¹H NMR (400 MHz, CDCl₃) δ 8.51 (d, J=1.6 Hz, 1H), 7.71 (s, 1H), 7.44-7.40 (m, 1H), 7.22-7.16 (m, 2H), 6.85 (d, J=8.0 Hz, 1H), 6.73 (d, J=7.8 Hz, 2H), 6.02 (s, 2H), 3.07 (s, 6H).

### Example 109: Synthesis of Compound 109

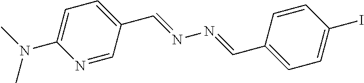 [SMILES: CN(C)c1ccc(/C=N/N=C/c2ccc(I)cc2)cn1]

According to the method for synthesizing compound 18, compound 109 was prepared from 6-dimethylaminonicotinaldehyde and 4-iodobenzaldehyde, and column chromatography separation was carried out using petroleum ether and ethyl acetate with a volume ratio of 4:1 as a developing solvent, to obtain 114.5 mg of light yellow crystalline solid with a yield of 30.3%. The structure was as follows: ¹H NMR (400 MHz, CDCl₃) δ 8.56 (s, 1H), 8.54 (s, 1H), 8.39 (d, J=2.2 Hz, 1H), 8.06 (dd, J=8.9, 1.5 Hz, 1H), 7.79-7.73 (m, 2H), 7.55-7.50 (m, 2H), 6.58 (d, J=9.0 Hz, 1H), 3.18 (s, 6H).

### Example 110: Synthesis of Compound 110

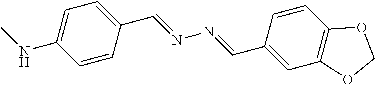 [SMILES: CNc1ccc(/C=N/N=C/c2ccc3c(c2)OCO3)cc1]

According to the method for synthesizing compound 18, compound 110 was prepared from 4-methylaminobenzaldehyde and piperonal, and column chromatography separation was carried out using petroleum ether and ethyl acetate with a volume ratio of 11:1 as a developing solvent, to obtain 33.4 mg of yellow crystalline solid with a yield of 11.9%. The structure was as follows: ¹H NMR (400 MHz, CDCl₃) δ 8.56 (s, 1H), 8.54 (s, 1H), 7.68 (d, J=7.8 Hz, 2H), 7.45 (d, J=1.7 Hz, 1H), 7.19 (dd, J=8.0, 1.5 Hz, 1H), 6.87-6.83 (m, 1H), 6.62 (d, J=8.8 Hz, 2H), 6.02 (d, J=4.1 Hz, 2H), 2.90 (s, 3H).

### Example 111: Synthesis of Compound 111

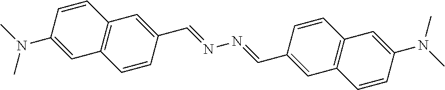

According to the method for synthesizing compound 1, compound 111 was prepared from 6-(dimethylamino)-2-naphthaldehyde, and 15.7 mg of yellow crystalline solid was obtained with a yield of 4.0%. The structure was as follows: ¹H NMR (400 MHz, CDCl₃) δ 8.85 (s, 2H), 8.04 (s, 4H), 7.79 (s, 2H), 7.71 (s, 2H), 7.21-7.12 (m, 2H), 6.99 (s, 2H), 3.12 (s, 12H).

### Example 112: Synthesis of Compound 112

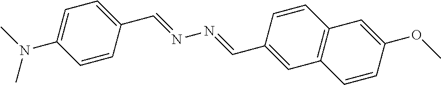

According to the method for synthesizing compound 18, compound 112 was prepared from 4-dimethylaminobenzaldehyde and 6-(methoxy)-2-naphthaldehyde, and column chromatography separation was carried out using petroleum ether and ethyl acetate with a volume ratio of 10:1 as a developing solvent, to obtain 50.0 mg of yellow crystalline solid with a yield of 14.8%. The structure was as follows: ¹H NMR (400 MHz, CDCl₃) δ 8.78 (s, 1H), 8.62 (s, 1H), 8.08-8.00 (m, 2H), 7.77 (dd, J=13.3, 8.8 Hz, 4H), 7.19-7.12 (m, 2H), 6.73 (d, J=9.0 Hz, 2H), 3.93 (s, 3H), 3.05 (s, 6H).

### Example 113: Synthesis of Compound 113

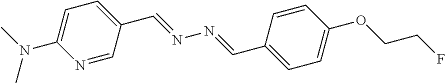 [SMILES: CN(C)c1ccc(/C=N/N=C/c2ccc(OCCF)cc2)cn1]

According to the method for synthesizing compound 18, compound 113 was prepared from 6-dimethyl aminonicotinaldehyde and 4-(2-fluoroethoxy)benzaldehyde, and column chromatography separation was carried out using petroleum ether and ethyl acetate with a volume ratio of 4:1 as a developing solvent, to obtain 27.0 mg of light yellow crystalline solid with a yield of 8.6%. The structure was as follows: ¹H NMR (400 MHz, CDCl₃) δ 8.58 (s, 1H), 8.57 (s, 1H), 8.39 (d, J=2.2 Hz, 1H), 8.07 (d, J=9.0, 2.0 Hz, 1H), 7.80-7.74 (m, 2H), 6.99 (t, J=5.9 Hz, 2H), 6.58 (d, J=9.0 Hz, 1H), 4.78 (dt, 2H), 4.27 (dt, 2H), 3.18 (s, 6H).

### Example 114: Synthesis of Compound 114

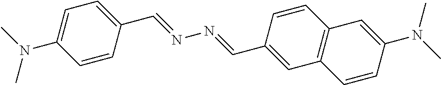

According to the method for synthesizing compound 18, compound 114 was prepared from 4-dimethylaminobenzaldehyde and 6-(dimethylamino)-2-naphthaldehyde, and column chromatography separation was carried out using dichloromethane, petroleum ether and ethyl acetate with a volume ratio of 10:5:4 as a developing solvent, to obtain 42.2 mg of yellow crystalline solid with a yield of 12.2%. The structure was as follows: ¹H NMR (400 MHz, CDCl₃) δ 8.75 (s, 1H), 8.60 (s, 1H), 7.95 (d, J=6.9 Hz, 2H), 7.74 (d, J=9.1 Hz, 3H), 7.65 (d, J=8.9 Hz, 1H), 7.15 (dd, J=9.1, 2.6 Hz, 1H), 6.90 (s, 1H), 6.73 (d, J=8.8 Hz, 2H), 3.08 (s, 6H), 3.05 (s, 6H).

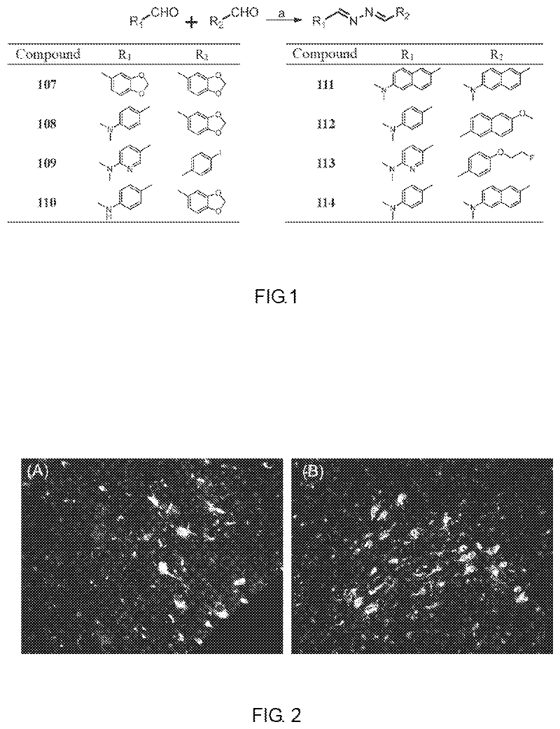

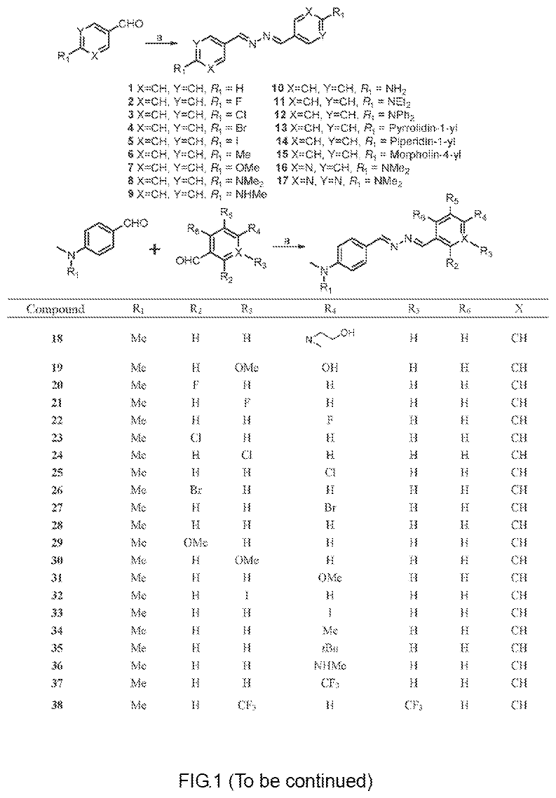

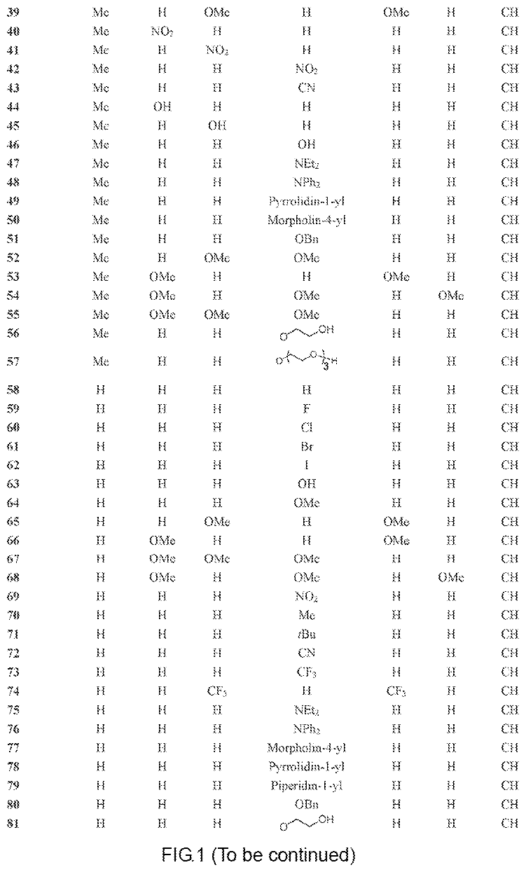

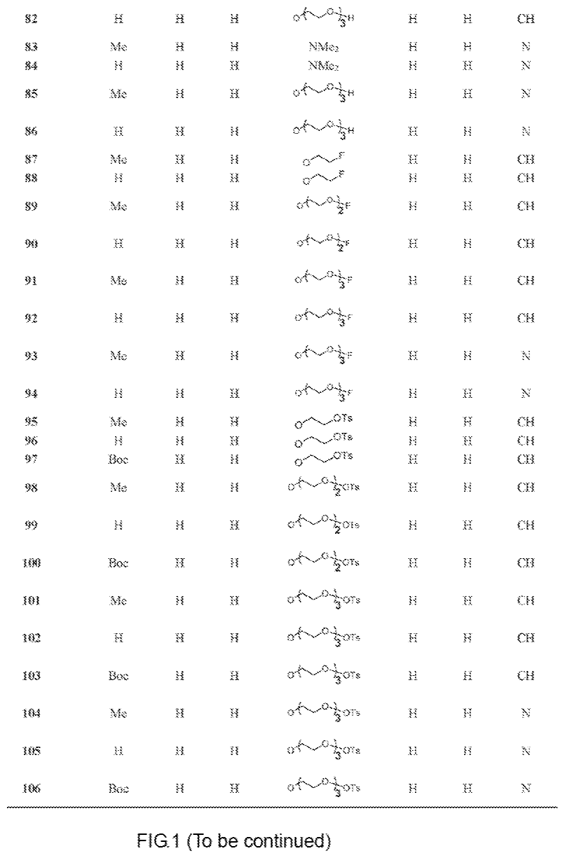

Schematic diagrams of the synthesis processes of the compounds of Examples 1-114 were shown in FIG. 1.

### Example 115: Preparation of ¹⁸F Labelled Ligand

1 Experimental Steps:

1) Preparation of Compounds [18F]87, [18F]88, [18F]89, [18F]90, [18F]91, [18F]92, [18F]93 and [18F]94

[18F] fluoride ions enriched in the QMA column were eluted into a 10 mL of glass reaction tube with 1.0 mL K222 eluent (a mixing solution containing 130 mg Kryptofix-2.2.2, 11 mg K₂CO₃, 8 mL acetonitrile, 2 mL water). The reaction tube is placed in a metal bath at 120° C. and heated, N₂ flow was filled from an open to volatilize the liquid in the tube. Then 1.0 mL of anhydrous acetonitrile was added in three times to azeotropically remove water with an interval of 3 minutes each time. After ensuring that the reaction system is anhydrous, 3.0 mg of labelled precursors, as intermediates 95, 96, 98, 99, 101, 102, 104 and 105, were dissolved in 500 μL anhydrous acetonitrile, and the precursor solution was transferred to the glass reaction tube containing [18F] fluoride ions. After the reaction tube was sealed, the reaction was carried out at a heating temperature of 100° C. for 6 minutes. After being cooled to room temperature, 500 μL of acetonitrile was added for dilution and then separation and purification were conducted by HPLC. The separation conditions were as follows: SilGreen MP C18 reverse column (5 μm, 10×250 mm), collecting the effluent of the labelled product, and removing the acetonitrile by rotary evaporation. The obtained product was dissolved in 10% ethanol aqueous solution for biological evaluation.

2 Experimental Results:

The labelling rates of [18F]87, [18F]88, [18F]89, [18F]90, [18F]91, [18F]92, [18F]93 and [18F]94 were all more than 40%, which is very advantageous to clinical transformation. After separation and purification by HPLC, the radiochemical purity is more than 95%, and corresponds to the retention time of the stable fluorinated ligand (Table 1).

TABLE 1
Retention time and purity of 18F-labelled ligand and its
stable ligand

| | | | chromato- | | |
|---|---|---|---|---|---|
| | | | graphic | | |
| | | Mobile | column | Retention | |
| com- | Flow rate | phase | (Venusil | time | purity |
| pound | (mL/min) | (CH3CN %) | MPC18) | (RT, min) | (%) |
| 87 | 4 | 50 | 10 × 250 mm | 9.088 | 100 |
| [18F]87 | 4 | 50 | 10 × 250 mm | 9.100 | 100 |
| 88 | 4 | 50 | 10 × 250 mm | 8.918 | 98.035 |
| [18F]88 | 4 | 50 | 10 × 250 mm | 8.934 | 99.010 |
| 89 | 4 | 50 | 10 × 250 mm | 15.221 | 98.425 |
| [18F]89 | 4 | 50 | 10 × 250 mm | 15.243 | 100 |
| 90 | 4 | 50 | 10 × 250 mm | 15.175 | 98.445 |
| [18F]90 | 4 | 50 | 10 × 250 mm | 15.316 | 100 |
| 91 | 4 | 50 | 10 × 250 mm | 14.572 | 99.940 |
| [18F]91 | 4 | 50 | 10 × 250 mm | 14.639 | 100 |
| 92 | 4 | 50 | 10 × 250 mm | 15.281 | 97.013 |
| [18F]92 | 4 | 50 | 10 × 250 mm | 15.339 | 100 |
| 93 | 4 | 45 | 10 × 250 mm | 12.635 | 97.480 |
| [18F]93 | 4 | 45 | 10 × 250 mm | 12.655 | 99.280 |
| 94 | 4 | 45 | 10 × 250 mm | 12.533 | 100 |
| [18F]94 | 4 | 45 | 10 × 250 mm | 12.547 | 100 |

### Example 116: Fluorescence Staining Experiment

1 Experimental Steps:

Fluorescence staining of pathological sections of brain tissue in AD patients:

(1) preparing an aqueous solution (containing 15% ethanol) with a concentration of 1 μM from target compound 8 or other probes to be used;

(2) soaking paraffin-embedded sections of the AD human brain with 8 μm thickness in xylene for 15 min to dewax, then rinsing with ethanol for 3×3 min, and rinsing with running water for 10 min, and placing them in 10 mM PBS (pH=7.4) for later use;

(3) dropping compounds to be tested onto the brain sections and incubating at room temperature for 15 min;

(4) after finishing incubation, washing the sections with 30% ethanol, sealing with resin, and observing under a fluorescence microscope.

2 Experimental results:

The experimental results were shown in FIG. 2. The probe designed by the present disclosure can clearly mark the neurofibrillary tangles NFTs located on AD human brain sections when used as an optical probe or a staining agent, indicating that the probe provided by the present disclosure can specifically bind to Tau protein.

### Example 117: Competitive Binding Experiment

1 Experimental Steps:

(1) formulating a 0.1% BSA solution with pH=7.4;

(2) preparing a radioligand [125I] IMPY according to the known methods; formulating [125I] IMPY into a solution of 60000-100000 cpm/100 μL for later use;

(3) formulating the compound to be tested into a 10⁻⁵ to 10⁻¹¹ mol/L serially diluted ethanol solution;

(4) preparing Aβ1-42 proteins according to the known methods, and diluting it into an aqueous solution of about 30 nM;

(5) soaking a glass fiber filter membrane in PBS solution containing 0.1% polyethyleneimine for 30 minutes;

(6) adding 100 μL of the test compound solutions with different concentrations and 100 μL of [125I] IMPY solution, 700 μL of BSA solution and 100 μL of Aβ1-42 solution into a 12 mm×75 mm borosilicate glass tube, and vortexing;

(7) incubating with shaking in a constant temperature water bath at 37° C. for 3 hours;

(8) collecting the reaction solution by a multi-head cell collector, and rinsing the reaction solution for three times with 10% ethanol solution;

(9) measuring and counting with a gamma counter;

(10) analyzing and processing data using GraphPrad Prism 5.01 to obtain an affinity constant Ki.

2 Experimental Results:

The experimental results were shown in Table 2. Activity between some compounds and Aβ₁₋₄₂ aggregates was quantitatively determined by the method. The results showed that this type of compound had a very good activity on Aβ₁₋₄₂ proteins.

TABLE 2
Quantitative activity data

| Compound | Ki (nM) | Compound | Ki (nM) |
|---|---|---|---|
| 9 | 45.17 ± 18.39 | 63 | 276.82 ± 50.53 |
| 11 | 5.10 ± 1.87 | 87 | 5.17 ± 1.40 |
| 13 | 1.20 ± 0.25 | 88 | 10.45 ± 6.71 |
| 14 | 1.39 ± 0.25 | 89 | 2.45 ± 1.70 |
| 15 | 147.05 ± 2.06 | 90 | 8.07 ± 2.89 |
| 16 | 6.46 ± 2.32 | 91 | 6.38 ± 0.72 |
| 17 | 123.71 ± 21.74 | 92 | 46.39 ± 15.52 |
| 36 | 35.09 ± 19.32 | 93 | 10.86 ± 0.97 |
| 94 | 42.89 ± 20.51 | | |

### Example 118: Autoradiography Experiment

After combining a certain concentration of the labelled products [¹⁸F]87-94 with the plaques in the brain sections of AD patients, they were exposed through a phosphor screen, and then the image was analyzed with a storage phosphor screen system.

1 Experimental Steps:

(1) preprocessing AD human brain sections;

(2) covering AD human brain sections with 100 μL of 20 μCi 18F-labelled compound solution, and incubating for 30 minutes at room temperature;

(3) rinsing with 40% ethanol solution for 1 minute;

(4) after drying, placing it under the phosphor screen and exposing for 30 minutes, and analyze the image with the storage phosphor screen system.

2 Experimental Results:

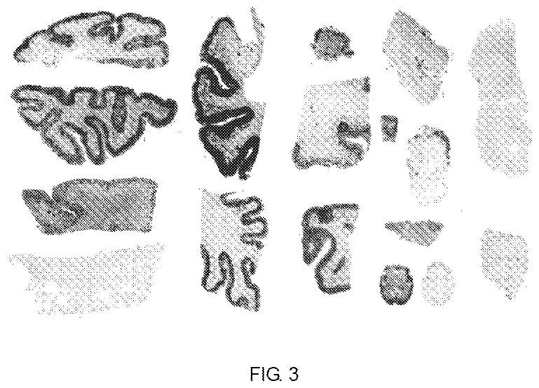

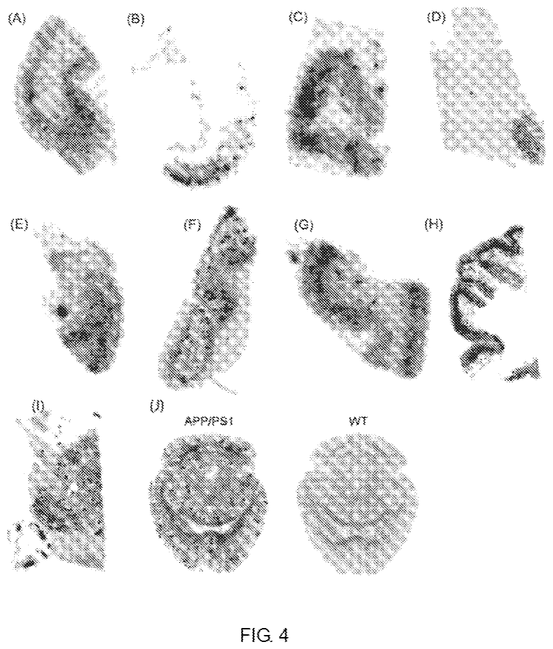

The experimental results were shown in FIGS. 3 to 4, fully demonstrating that the compounds of the present disclosure can locate Aβ protein in AD brain with high resolution after being labelled with radionuclide, and thus are promising Aβ imaging agents in clinical diagnosis.

### Example 119: Biodistribution Experiment in Normal Mice

Pharmacokinetic properties in mice, especially the initial brain uptake and brain clearance, were studied through distribution experiments in vivo.

1 Experimental Steps:

5-10 μCi labelled compounds (100 μL physiological saline solution, containing 10% ethanol) were injected into normal mice (ICR, male, 20-22 g, 5 weeks old) through the tail vein (n=5), and mice were decapitated at 2 minutes, 10 minutes, 30 minutes and 60 minutes after injection, respectively, and organs to be tested were took out by dissection, and the wet weight was measured and radioactive counting was conducted. Data is expressed as the percentage of radioactive dose per gram of organ (% ID/g).

2 Experimental Results:

The experimental results were shown in Table 3. The probes [18F]87, [18F]88, [18F]89, [18F]90, [18F]91, [18F]92, [18F]93 and [18F]94 recited in the present disclosure can effectively pass through the blood-brain barrier, and the brain uptake reaches its peak at 2 minutes and are cleared faster in the brain of normal mice.

TABLE 3
Results of biodistribution of ¹⁸F labelled compounds in
normal miceⁿ

| Compound | Organ | 2 min | 10 min | 30 min | 60 min |
|---|---|---|---|---|---|
| [18F]87^{a} | blood | 3.98 ± 0.24 | 3.36 ± 0.19 | 3.00 ± 0.13 | 2.73 ± 0.42 |
| | brain | 10.66 ± 0.89 | 11.00 ± 0.80 | 6.69 ± 0.42 | 4.56 ± 0.58 |
| [18F]88^{a} | blood | 3.32 ± 0.26 | 3.57 ± 0.34 | 3.31 ± 0.13 | 2.90 ± 0.32 |
| | brain | 9.21 ± 0.63 | 8.24 ± 0.36 | 4.81 ± 0.27 | 3.02 ± 0.15 |
| [18F]89^{a} | blood | 2.95 ± 0.31 | 3.24 ± 1.25 | 4.78 ± 0.24 | 5.42 ± 0.65 |
| | brain | 10.88 ± 0.75 | 10.62 ± 0.58 | 6.06 ± 0.38 | 4.55 ± 0.48 |
| [18F]90^{a} | blood | 3.72 ± 0.44 | 3.85 ± 0.26 | 4.90 ± 0.61 | 5.44 ± 0.40 |
| | brain | 11.05 ± 1.70 | 7.81 ± 0.60 | 4.92 ± 0.41 | 3.97 ± 0.28 |
| [18F]91^{a} | blood | 3.08 ± 0.45 | 2.86 ± 0.28 | 3.83 ± 0.27 | 3.66 ± 0.40 |
| | brain | 10.39 ± 1.07 | 7.56 ± 0.91 | 2.93 ± 0.13 | 1.60 ± 0.10 |
| [18F]92^{a} | blood | 3.81 ± 0.35 | 3.06 ± 0.49 | 3.26 ± 0.17 | 3.05 ± 0.45 |
| | brain | 7.84 ± 0.50 | 3.94 ± 0.13 | 1.88 ± 0.03 | 1.17 ± 0.07 |
| [18F]93^{a} | blood | 3.53 ± 0.24 | 3.16 ± 1.00 | 3.64 ± 0.29 | 2.46 ± 0.33 |
| | brain | 11.39 ± 1.08 | 5.55 ± 0.37 | 2.41 ± 0.22 | 1.44 ± 0.15 |
| [18F]94^{a} | blood | 4.30 ± 0.09 | 3.40 ± 0.28 | 3.66 ± 0.18 | 2.59 ± 0.15 |
| | brain | 8.59 ± 0.57 | 4.09 ± 0.38 | 2.40 ± 0.15 | 1.36 ± 0.13 |

^{a} is expressed as % ID/g, and n is the number of experimental mice.

Although the general description and specific embodiments have been used to describe the present disclosure in detail above, it is obvious to a person skilled in the art that some modifications or improvements can be made on the basis of the present disclosure. Therefore, these modifications or improvements made without departing from the spirit of the present disclosure belong to the protection scope of the present disclosure.

### INDUSTRIAL APPLICABILITY

The present disclosure provides dihydrazone compounds having high affinity to Aβ protein and Tau protein and derivatives thereof. The structure of the compounds is as shown by formula (I):

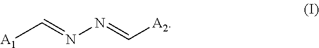

Such compounds can be directly used as fluorescent probes for detecting neurofibrillary tangles in vivo or in a tissue sample, and when used for nuclear medicine imaging, the compounds need to be labelled with appropriate radioisotopes. Such compounds are particularly suitable for diagnosing patients with neurodegenerative diseases, including Alzheimer's disease, having the characteristics of Aβ plaques and neurofibrillary tangles, which have good economic values and application prospects.

1. A dihydrazone compound having high affinity to Aβ protein and Tau protein, or pharmaceutically acceptable salts, esters, or amides thereof, wherein a structure of the compound is shown in formula (I): 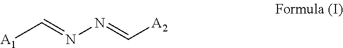
wherein, A₁ and A₂ each independently represent an aryl group or a substituted aryl group;
the aryl group or substituted aryl group is selected from 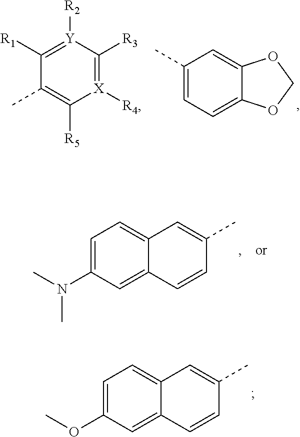
X and Y each independently represent C or N, wherein when X is N, R₄ does not exist, and when Y is N, R₂ does not exist;
R₁, R₂, R₃, R₄, and R₅ each independently represent H, ¹⁸F, F, Cl, Br, ¹²³I, ¹²⁴I, ¹²⁵I, I, CH₃, tBu, NO₂, CN, CF₃, OH, O¹¹CH₃, OCH₃, NH₂, NH¹¹CH₃, NHCH₃, N(CH₃)₂, N(CH₂CH₃)₂, N(Ph)₂, 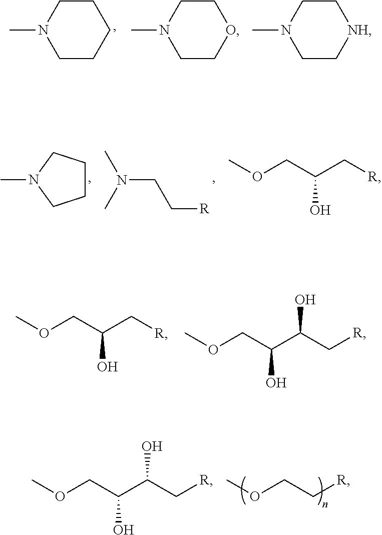
O(CH₂)ₘ¹⁸F, O(CH₂)ₘ¹⁹F; wherein R is OH, ¹⁸F or ¹⁹F, m and n are integers between 1-6.

2. The compound according to claim 1, wherein the compound is selected from any one of the following compounds 8, 9, 11, 13-17, 36, 94, 63, and 87-93:

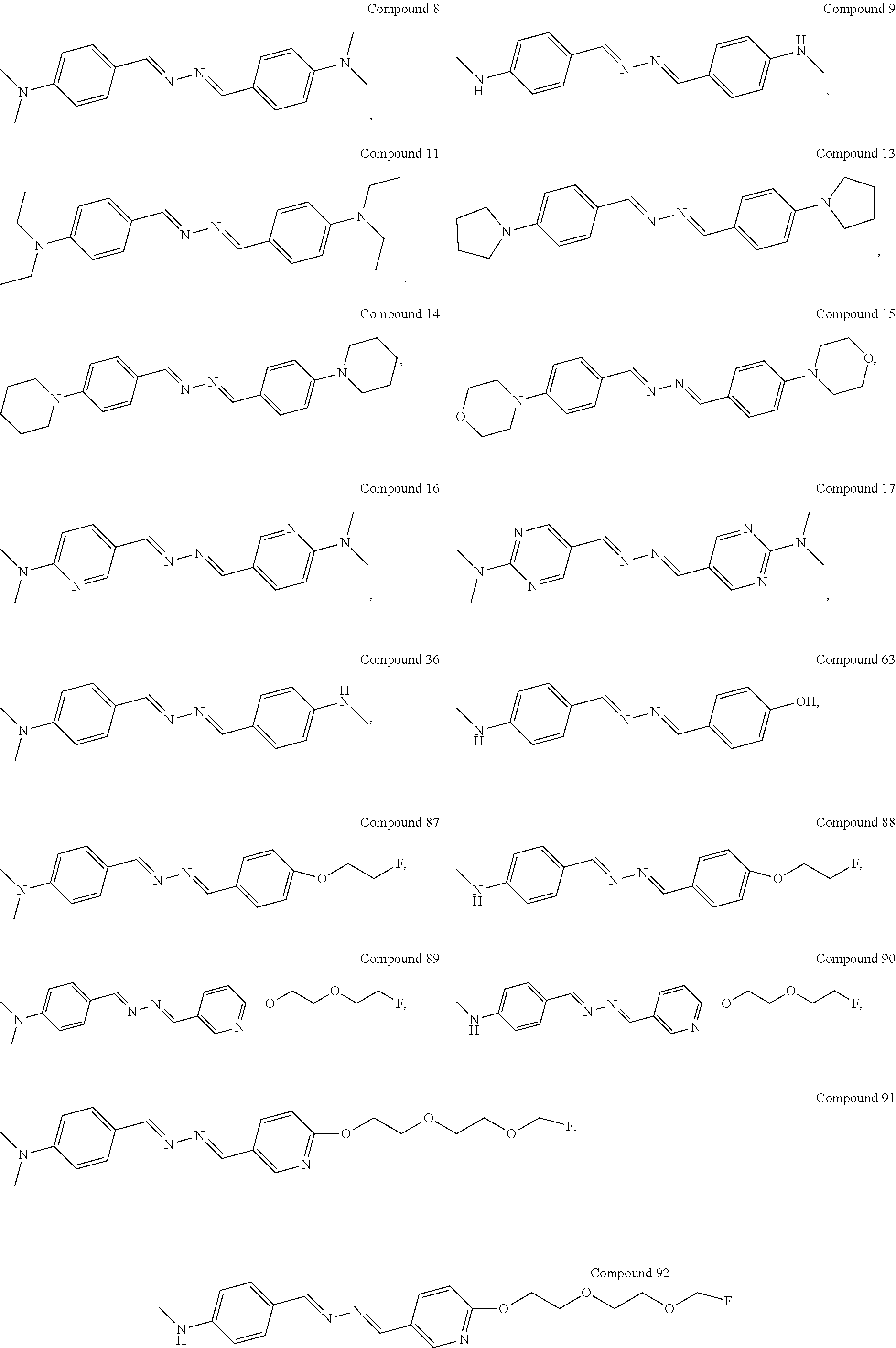

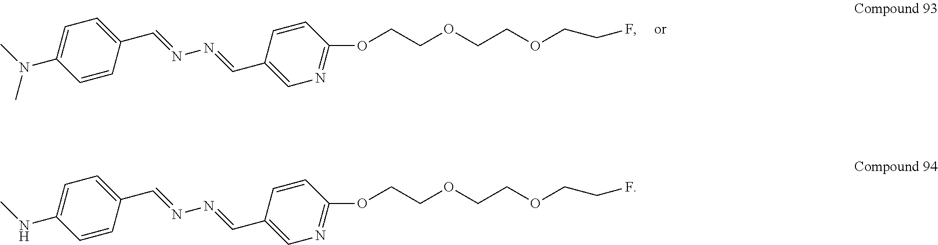 [SMILES: CN(C)c1ccc(/C=N/N=C/c2ccc(OCCOCCOCCF)nc2)cc1 ; CNc1ccc(/C=N/N=C/c2ccc(OCCOCCOCCF)nc2)cc1]

3. The compound according to claim 2, wherein the compound is compounds 87-94.

4. (canceled)

5. A diagnostic or detecting reagent for neurodegenerative diseases caused by Aβ protein or Tau protein deposition, wherein an active ingredient is the compound of claim 1 and/or pharmaceutically acceptable salts, esters, or amides of the compound of claim 1.

6. The diagnostic or detecting reagent according to claim 5, wherein the diseases is Alzheimer's disease, frontotemporal degeneration, chronic traumatic encephalopathy, progressive supranuclear palsy, cortical basal ganglia degeneration or Pick's disease.

7. A nuclear medicine imaging agent, optical imaging agent or staining agent comprising the compound of claim 1 and/or pharmaceutically acceptable salts, esters, or amides of the compound of claim 1.
